SEQUENCE LISTING (7 sequences)

SEQ ID NO: 1 - RNA, 20 nt, synthetic construct
  source 1..20: mol_type = other RNA
gcgcgtatcatcccttaccg

SEQ ID NO: 2 - RNA, 20 nt, synthetic construct
  source 1..20: mol_type = other RNA
cgccgtatcatcccttaccg

SEQ ID NO: 3 - RNA, 26 nt, synthetic construct
  source 1..26: mol_type = other RNA
cgcgctgcgcgtatcatcccttaccg

SEQ ID NO: 4 - RNA, 27 nt, synthetic construct
  source 1..27: mol_type = other RNA
acgcgctgcgcgtatcatcccttaccg

SEQ ID NO: 5 - RNA, 28 nt, synthetic construct
  source 1..28: mol_type = other RNA
tacgcgctgcgcgtatcatcccttaccg

SEQ ID NO: 6 - RNA, 26 nt, synthetic construct
  source 1..26: mol_type = other RNA
atattggcgcgtatcatcccttaccg

SEQ ID NO: 7 - RNA, 42 nt, synthetic construct
  source 1..42: mol_type = other RNA
ggtaatttctactcttgtagatgccattccctactatgcgcg

(19) United States
(12) Patent Application Publication
(10) Pub. No.: US 2025/0011769 A1
(43) Pub. Date: Jan. 9, 2025

(54) MODULATION OF THE CRISPR ROADBLOCK

(71) Applicant: Cornell University, Ithaca, NY (US)

(72) Inventors: Michelle D. WANG, Ithaca, NY (US); Porter M. HALL, Ithaca, NY (US)

(21) Appl. No.: 18/709,939

(22) PCT Filed: Nov. 17, 2022
(86) PCT No.: PCT/US2022/080085

§ 371 Date: May 14, 2024

Related U.S. Application Data
(60) Provisional application No. 63/280,448, filed on Nov. 17, 2021.

(51) Int. Cl. C12N 15/11; C12N 9/22

(52) U.S. Cl. CPC C12N 15/111; C12N 9/22; C07K 2319/09; C12N 2310/20; C12N 2310/3519; C12N 2320/52

(57) ABSTRACT

Provided are modified guide RNAs (gRNAs) for use with CRISPR Cas proteins. A modified guide RNA comprises at its 5′ or 3′end at least 5 nucleotides that comprise an inverted repeat sequence having a segment targeted to a spacer sequence in DNA. The inverted repeat sequence is configured so that it can concurrently be hybridized to the spacer sequence and to the complementary strand of the DNA comprising the spacer sequence when in the presence of the DNA and the Cas protein. The modified gRNA influences the Cas protein interaction with DNA.

### CROSS-REFERENCE TO RELATED APPLICATIONS

This application claims priority to U.S. provisional patent application No. 63/280,448, filed Nov. 17, 2021, the entire disclosure of which is incorporated herein by reference.

### STATEMENT REGARDING FEDERALLY SPONSORED RESEARCH OR DEVELOPMENT

This invention was made with government support under grant number R01GM136894 awarded by National Institutes of Health, and grant number T32GM008267 awarded by the National Science Foundation. The government has certain rights in the invention.

### SEQUENCE LISTING

The instant application contains a Sequence Listing which has been submitted in.xml format and is hereby incorporated by reference in its entirety. Said.xml file is named “018617_01388_ST26”, was created on Nov. 17, 2022, and is 7,089 bytes in size.

### FIELD

The present disclosure relates generally to the function of Cas nucleases and more specifically to guide RNAs that affect aspects of DNA binding by Cas nucleases.

### BACKGROUND

The utilization of CRISPR-associated (Cas) nucleases offers the ability to precisely target DNA sequences and cleave at those sites, enabling great advances in gene editing, targeting, and diagnostic technology for both prokaryotic and eukaryotic systems¹⁴. To accomplish this, a Cas protein is complexed with a guide RNA (gRNA) that contains a spacer region complementary to the target DNA sequence. A facet of CRISPR utility relies on Cas enzyme binding stability which is dictated by specific and robust binding of the gRNA to the target DNA sequence. This occurs via recognition of a protospacer adjacent motif (PAM) sequence and hybridization of the spacer region of the gRNA with the target DNA to form a gRNA/DNA hybrid (R-loop)¹⁵.

In vivo, a DNA-bound Cas can not only dissociate from the DNA spontaneously but also be removed by motor proteins carrying out other host processes. However, the mechanism governing Cas removal by motor proteins is not well-understood. Intriguingly, during CRISPR interference (CRISPRi), which uses an endonuclease-deficient Cas (dCas) to block transcription, the effectiveness of dCas removal depends on the orientation of the bound dCas relative to transcription. Transcription elongation is rather permissive from the PAM-distal side of a bound dCas but is predominantly blocked from the PAM-proximal side⁶⁻⁸. Curiously, a bound dCas is not found to be a polar barrier to replication^{9,10}, indicating that the polarity is dictated by the dynamics of how motor proteins overcome dCas barriers. Thus, there is an ongoing and unmet need to elucidate how Cas proteins interact with DNA and influence Cas protein DNA binding, and to provide compositions and methods to influence Cas protein binding to DNA in the CRISPR context. The present disclosure is pertinent to this need.

### BRIEF SUMMARY

The present disclosure provides modified guide RNAs (gRNAs) for use with CRISPR Cas proteins. A modified guide RNA comprises at its 5′ or 3′ end at least 5 nucleotides that comprise an inverted repeat sequence having a segment targeted to a spacer sequence in DNA. The inverted repeat sequence is configured so that it can concurrently be hybridized to the spacer sequence and to the complementary strand of the DNA comprising the spacer sequence when in the presence of the DNA and the Cas protein. In certain embodiments, the inverted repeat comprises 5, 6, or 7 nucleotides. In non-limiting examples, the CRISPR Cas protein comprises a nuclease dead protein. The disclosure also provides expression vectors encoding the modified gRNAs, which may also encode one or more Cas proteins. The disclosure also provides a ribonucleoprotein comprising a CRISPR Cas protein and a modified guide RNA as described herein.

In embodiments, the disclosure provides a method comprising introducing into cells a described modified guide RNA and a Cas protein so that a complex comprising the modified guide RNA, the DNA and the Cas protein forms within the cell. The complex is such that the inverted repeat sequence is concurrently hybridized to the spacer sequence and to the complementary strand of the double stranded DNA, and is in association with the Cas protein.

To provide the foregoing embodiments, the disclosure describes a single-molecule assays used to map structural features of a dCas complex bound to DNA and analysis of how an elongating RNA polymerase (RNAP) interacts with the bound dCas. This description is extendable to other motor proteins that are double stranded DNA translocases. Through this analysis, the disclosure provides a description of the mechanism for CRISPR interference (CRISPRi) polarity and dCas removal, demonstrating influence of the R-loop stability for a bound Cas. This mechanistic understanding supports compositions and methods for modulating dCas and is applicable to modulating the function of other Cas proteins that interact with DNA. In embodiments, the disclosure demonstrates that modulating the dCas R-loop stability by using modified gRNAs can improve Cas protein resistance to removal from the DNA by motor proteins. Thus, use of the described modified gRNAs enables modulation of Cas protein function when the Cas protein is present in a complex comprising a modified gRNA and DNA.

### BRIEF DESCRIPTION OF THE FIGURES

FIG. 1. High-resolution maps of dCas interactions with DNA using the DNA unzipping mapper. Panel a, DNA unzipping mapper configuration. An unzipping template is tethered at one end to the surface of a coverslip of a sample chamber and at the other end to a polystyrene bead held in an optical trap. Using an optical trap, the bead is moved relative to the surface, progressively unzipping the DNA until the unzipping fork reaches a bound protein, which resists unzipping, leading to a distinct force rise. The location of the force rise is used to map the protein location. Panel b, Shown are representative unzipping traces (red) of bound dCas9 (top), bound dCas12a (middle), and a paused transcription elongation complex (TEC) (bottom), along with naked DNA traces (black). The DNA was unzipped from either direction (black arrows) relative to the bound protein for each protein. Two conformations were detected when a bound dCas 12a protein was unzipped from the PAM distal side, shown as light blue and red. The two dashed lines bracket the expected gRNA/DNA hybrid locations for dCas9 or dCas12a and the expected RNA/DNA hybrid of a TEC. Red arrows indicate locations where the unzipping force dipped below the naked DNA baseline. Panel c, Hypothesized mechanism for transcription read-through from the PAM-distal side. Note that gRNA hybridizes with the TEC template and non-template strand for a bound dCas9 and dCas12a complex, respectively.

FIG. 2. A quantitative assay for transcription read-through of a bound dCas complex. Panel a, Flowchart of a single molecule transcription assay for a given sample chamber. Some DNA tethers were used as controls to assay bound protein locations prior to NTP addition. Other tethers were used to assay bound protein locations after an NTP chase time of Δt=135 s. DNA was always unzipped in the same direction as RNAP translocation. Panel b, Representative traces of RNAP encountering a bound dCas9 from the PAM-distal side. An example control trace is shown with RNAP and bound dCas9 detected at their expected locations. After NTP addition, shown are example traces of RNAP colliding with dCas9 and reading through dCas9. Naked DNA traces are shown in black. Panel c, Transcription read-through efficiency for RNAP encountering a bound dCas from either the PAM-distal side or the PAM-proximal side. Results from both dCas9 (top) and dCas12a (bottom) are shown. For each sample chamber, both control traces and non-control traces were taken to obtain the read-through efficiency for that chamber. Each type of experiment was repeated using N biologically independent sample chambers: dCas9 PAM-distal, N=6 (−GreB) and N=6 (+GreB); dCas9 PAM-proximal, N=5 (−GreB) and N=5 (+GreB); dCas12a PAM-distal, N=5 (−GreB) and N=5 (−GreB); dCas12a PAM-proximal, N=6 (−GreB) and N=5 (+GreB). Read-through values were calculated for each sample chamber (black dots) and the mean and SEM of these repeats are also shown.

FIG. 3. Mfd moving through a bound dCas complex. Panel a, Flowchart of a single molecule Mfd translocation assay. Some DNA tethers were used as controls to assay bound protein locations prior to ATP addition. Other tethers were used to assay bound protein locations after an ATP chase time of Δr=8 min. During the ATP chase, Mfd was able to translocate along DNA, likely still interacting with a non-transcribing RNAP. DNA was always unzipped in the same direction as Mfd translocation. Panel b, Representative traces of Mfd colliding with dCas9 and moving past dCas9, when approaching dCas9 from the PAM-distal side. Naked DNA traces are shown in black. Panel c, Mfd move-through efficiency for Mfd encountering a bound dCas from either the PAM-distal side or the PAM-proximal side. Results from both dCas9 (top) and dCas12a (bottom) are shown. For each sample chamber, both control traces and non-control traces were taken to obtain the move-through efficiency for that chamber. Each type of experiment was repeated using N=6 biologically independent sample chambers. Move-through values were calculated for each sample chamber (black dots), and the mean value and SEM of these repeats are also shown.

FIG. 4. Modulation of transcription read-through of a bound dCas complex via gRNA modifications. Panel a, Cartoons depicting the four types of modified gRNA. Panel b, Representative unzipping mapper traces that highlight the force signature difference between a bound dCas9 containing a modified gRNA with a 7-nt inverted repeat (IR) and a bound dCas9 containing an unmodified gRNA. Vertical dashed lines bracket the position of the gRNA/DNA hybrid. Naked DNA traces are shown in black. The x-axis arrow indicates the location of the unzipping force dropping below the naked DNA baseline for the trace with an unmodified gRNA. Panel c, Transcription read-through efficiency for RNAP encountering a bound dCas from either the PAM-distal side or the PAM-proximal side. DNA was always unzipped in the same direction as RVAP translocation. For each sample chamber, both control traces and non-control traces were taken to obtain the read-through efficiency for that chamber. Each type of experiment was repeated using N biologically independent sample chambers: dCas9 PAM-distal, N=6 (3-nt mismatch), N=6 (unmodified), N=5 (5-nt IR), N=8 (6-nt IR), and N=8 (7-nt IR); dCas9 PAM-distal, N=5 (3-nt mismatch), N=5 (unmodified); N=5 (5-nt IR), N=6 (6-nt IR), and N=5 (7-nt IR). Read-through values were calculated for each sample chamber (black dots), and the mean value and SEM of these repeats are also shown.

FIG. 5. dCas removal mediated by RNAP invasion of the dCas R-Loop. Panel a, RNAP collision with dCas9 from the PAM-proximal side without dCas9 removal. (Top) An example unzipping trace. (Bottom) Force peak locations of RNAP and dCas9. Dashed lines indicate expected RNAP force peak position when RNAP comes into contact with dCas9 (FIG. 7 Panel a). Each box plot represents the 25^{th}-75^{th} percentiles of the force peak positions of N biologically independent traces with error bars indicating SDs: N=88 (3-nt mismatch), N=114 (unmodified), N=103 (5-nt IR), N=108 (6-nt IR), and N=103 (7-nt IR). Panel b, RNAP collision with dCas9 from the PAM-distal side without dCas9 removal. (Top) An example unzipping trace showing the stalled RNAP and dCas9 force peaks. (Bottom) Force peak locations of RNAP after collision with dCas9. Each box shows the 25^{th}-75^{th} percentiles of the force peak position distribution with error bars indicating SDs: N=14 (3-nt mismatch), N=77 (unmodified), N=83 (5-nt IR), N=157 (6-nt IR), and N=163 (7-nt IR). Panel c, RNAP collision with dCas9 from the PAM-distal side with dCas9 being removed. (Top) An example unzipping trace showing the stalled RNAP force peak with the dCas9 force peak being absent. (Bottom) Force peak locations of RNAP and dCas9. Each box shows the 25^{th}-75^{th} percentiles of the force peak position distribution with error bars indicating SDs: N=23 (3-nt mismatch), N=22 (unmodified), N=12 (5-nt IR), N=15 (6-nt IR), and N=12 (7-nt IR). Panel d. The efficiency of dCas9 removal with different gRNA modifications. Out of the removal efficiency, the percent of traces with removal due to transcription read-through (grey, from FIG. 4 Panel c) and the percent from traces with removal but without read-through (red) are stacked. Percent dCas-removal values were calculated for each sample chamber (black dots), and the mean value and SEM of these repeats are also shown. The same data used for FIG. 4 Panel c were used for this analysis, so the sample statistics are identical to those for FIG. 4 Panel c.

FIG. 6. Mechanism of CRISPR roadblock polarity to transcription. When RNAP encounters a bound dCas from the PAM-distal side, RNAP may rezip the DNA bubble of the bound dCas, leading to R-loop disruption and DNA bubble collapse. The subsequent dCas removal allows transcription read-through. In contrast, when RNAP encounters a bound dCas from the PAM-proximal side, the DNA bubble of the bound dCas is not directly accessible to RNAP, making the dCas a strong barrier to transcription. Note that this mechanism is illustrated using dCas9 cartoon, but the same mechanism also applies to dCas12a.

FIG. 7. RNAP encountering a bound dCas protein from the PAM-proximal side. Panel a, Structural features of TEC and dCas. Numbers shown are our best estimates based on published structural data of dCas9^{18,21,23,64}, dCas12a^{57,65}, and TEC^{29,55,66} Panel b, Cartoon of RNAP approaching the PAM proximal region of dCas9 or dCas12a. Panel c, The distribution of stall forces of an actively elongating RNAP obtained using the unzipping staller method²⁶ is compared to the peak disruption forces from PAM-proximal unzipping of dCas proteins using the unzipping mapper technique from FIG. 1. The forces required to disrupt a bound dCas from the PAM-proximal dCas side are well above the forces that RNAP can generate working against a fork before stalling. This suggests that the dCas barrier from the PAM-proximal side is unsurpassable by RNAP.

FIG. 8. Transcription and dCas binding control experiments. Panel a, Flowchart of the single-molecule assay for a control experiment to determine RNAP speed and processivity. Panel b, The mean distance RNAP traveled as a function of chase time. N number of biologically independent traces were used for analysis at each time point: N=42 at 65 s, N=44 at 90 s, N=65 at 110 s, and N=18 at 120 s. Error bars are SEMs. The gray dashed line is a linear fit, yielding a slope of 15.4±0.6 bp/s for the RNAP speed. Panel c, Percent of RNAP remaining on the template normalized to initial occupancy. N number of biologically independent traces were used for analysis at each time point: N=58 at 65 s, N=65 at 90 s, N=104 at 110 s, and N=34 at 120 s. Error bars are SEMs. The average 96% occupancy (grey dashed line) indicates that around 4% of RNAP likely dissociated upon chasing before transcription resumption, but the remaining population remained bound as RNAP moved down the template. Panel d, Flowchart of the single-molecule assay for a control experiment to measure the fraction of dCas remaining bound after the introduction of transcription conditions. Panel e, % of dCas remaining bound after the quench. For each sample chamber, both control traces and non-control traces were taken to obtain the % dCas remaining for that chamber. Each type of experiment was repeated using N biologically independent sample chambers: Cas9, N=4 (3-nt mismatch), N=4 (unmodified), N=3 (5-nt IR), N=3 (6-nt IR), and N=3 (7-nt IR); dCas12a, N=4 (unmodified). % dCas remaining values were calculated for each sample chamber (black dots), and the mean value and SEM of these repeats are also shown.

FIG. 9. RNAP locations upon collisions with a bound dCas protein for data shown in FIG. 2. Distributions of RNAP force peak locations after NTP addition for dCas9 (Panel a) or dCas12a (Panel b) for each condition in FIG. 2. RNAP locations were determined after quenching transcription assays with a PAM-distal (top) or PAM-proximal (bottom) bound dCas complex. Each dCas orientation was assayed either in the presence or the absence of GreB during chasing. The expected locations of the A20 and the PAM site are indicated as dashed lines. The RNAP force peak locations were pooled for N biologically independent traces: dCas9 PAM-distal, N=217 (−GreB) and N=184 (+GreB); dCas9 PAM-proximal, N=174 (−GreB) and N=190 (−GreB); dCas12a PAM-distal, N=160 (−GreB) and N=177 (+GreB); dCas12a PAM-proximal, N=214 (−GreB) and N=192 (+GreB).

FIG. 10. Representative traces of transcription encountering dCas9 from the PAM proximal side and encountering dCas 12a from both sides. Panel a, Flowchart of the transcription read-through assay. Panel b, Representative traces of RNAP encountering a bound dCas9 from the PAM proximal side. An example control trace is shown with RNAP and bound dCas9 detected at their expected locations. After NTP addition, shown are example traces of RNAP prior to encountering the dCas9 (top) and RNAP colliding with dCas9 (bottom). Panel c, Representative traces of transcription assays with dCas12a in the PAM-Distal orientation. An example control trace is shown with RNAP and bound dCas12a detected at their expected locations. After NTP addition, shown are example traces of RNAP prior to encountering the dCas12a (top), RNAP colliding with dCas12a (middle), and RNAP reading through dCas12a (bottom).

FIG. 11. Trace classification for transcription assays. Cartoons represent the observed states in a sample chamber before chase (left) and after chase (right). Arrows indicate possible transitions between initial and final states. This diagram informs equations that represent different pathways for transitions between the initial and final states as described in methods and is used to solve for the relevant parameters of read-through and dCas removal.

FIG. 12. Read-through polarity of dCas9 and dCas 12a via bulk transcription. Shown are results from bulk transcription run-off assays with bound dCas9 (Panel a) or dCas12a (Panel b) proteins in either the PAM-distal or PAM-proximal orientation relative to the promoter. Transcription was carried out with 1 mM NTPs in the presence or absence of 1 μM GreB for 135 s before being quenched with formamide and EDTA to stop the reaction. Transcripts were assayed by 6% denaturing PAGE gels (Methods). The distance from the +1 to the PAM sequence for the PAM-distal orientation collision was 349 bp for both dCas9 and dCas12a, while this distance for the PAM-proximal collision was 332 bp for dCas9 and 333 bp for dCas12a. The locations of the A20, dCas collision, and run-off products are indicated with arrows. The transcription read-throughs from these gels were ˜35% without GreB and ˜77% with GreB for PAM-distal dCas9 collisions, ˜6% without GreB and ˜6% with GreB for PAM-proximal dCas9 collisions, ˜31% without GreB and ˜54% with GreB for PAM-distal dCas12a collisions, and ˜9% without GreB and ˜8% with GreB for PAM-distal dCas12a collisions. We performed an additional transcription gel assay for each dCas complex, and those gels yielded similar results as shown here.

FIG. 13. Mfd speed and processivity. Panel a, Flowchart of the Mfd only control experiments. Sample chambers containing TEC paused at 20 are formed as in FIGS. 2-5, and dCas was not bound the DNA. In contrast to FIG. 3, tethers were unzipped while Mfd was translocating to assess Mfd moving in “real-time.” Panel b, Mfd translocation versus time. N number of biologically independent traces were used for analysis at each time point: N=2 at 0 s, N=10 at 90 s, N=21 at 180 s, N=25 at 270 s, N=11 at 360 s, and N=6 at 450 s. Error bars are SEMs for the vertical axis and SDs for the horizontal axis. The black dashed line is a linear fit, giving a speed of 2.2±0.35 bp/s. Panel c, % Mfd remaining bound versus time. Each data point is normalized against the fraction of tethers initially containing an Mfd. N number of biologically independent traces were used for analysis at each time point: N=5 at 72 s, N=25 at 137 s, N=30 at 225 s, N=31 at 310 s, N=31 at 398 s, N=15 at 476 s. Error bars are SEMs.

FIG. 14. RNAP locations upon collisions with a bound dCas protein for data shown in FIGS. 4 and 5. Distributions of RNAP force peak locations after NTP addition for each condition in FIGS. 4-5. The locations of the A20 and the PAM site are indicated with dashed lines. RNAP locations were determined after quenching transcription assays with a PAM-distal (left) or PAM-proximal (right) bound dCas complex. The RNAP force peak locations were pooled for N biologically independent traces: dCas9 PAM-distal, N=150 (3-nt mismatch), N=217 (unmodified), N=151 (5-nt IR), N=282 (6-nt IR), N=295 (7-nt IR); dCas9 PAM-proximal, N=165 (3-nt mismatch), N=174 (unmodified), N=175 (5-nt IR), N=169 (6-nt IR), and N=167 (7-nt IR).

FIG. 15. Transcription read-through and removal efficiency of a bound dCas9 complexed with gRNA containing a 6-nt extension, which is not complementary to the target DNA. Panel a, % of dCas remaining bound after the quench. A control experiment to measure the fraction of dCas9 containing this modified gRNA (Supplementary Table 2) remaining bound after the introduction of the transcription conditions, using the method described in FIG. 2. For each sample chamber, both control traces and non-control traces were taken to obtain the % dCas remaining for that chamber. The experiment was repeated using N=3 biologically independent sample chambers. The % dCas remaining value was calculated for each sample chamber (black dots), and the mean value and SEM of these repeats are also shown. Panel b, The transcription assay was performed using the modified gRNA for both the PAM-distal and PAM-proximal orientations. For each sample chamber, both control traces and non-control traces were taken to obtain the % dCas removal for that chamber. The experiment was repeated using N=6 biologically independent sample chambers. The % dCas removal value was calculated for each sample chamber (black dots), and the mean value and SEM of these repeats are also shown.

FIG. 16. dCas9 and dCas 12a removal efficiency during transcription in the presence of GreB. The same data taken for FIG. 2 Panel c were used for this analysis, so the sample trace statistics are identical to those for FIG. 2 Panel c. Out of the removal efficiency, the fraction from traces with removal due to transcription read-through (grey) and the fraction from traces with removal but without transcription read-through (red) are stacked. Black dots are removal efficiencies calculated for each independent replicate. Error bars are SEMs.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

The amino acid or polynucleotide sequence as the case may be associated with each GenBank or other database accession number of this disclosure is incorporated herein by reference as presented in the database on the effective filing date of this application or patent.

The disclosure provides modified gRNAs that are functional with a Cas protein. By “functional” it is meant that the gRNA is capable of targeting the Cas protein to a DNA target comprising a sequence that is complementary to a spacer sequence in the gRNA.

The disclosure includes all modified gRNAs in the form as described herein, i.e., a gRNA comprising an appended inverted repeat sequence. The inverted repeat sequence comprises at least 5 nucleotides. In embodiments, the inverted repeat sequence comprises or consists of 5, 6, or 7 nucleotides, although longer or shorter inverted repeat sequences may be included. The term “repeat” in the described inverted repeat sequence does not mean a repeat sequence in a CRISPR array. A nucleotide that is not part of the inverted repeat sequence may be present between the inverted repeat and the spacer sequence. Any modified gRNA described herein may be a single guide RNA such that includes trans-activating CRISPR RNA (tracrRNA) and a crRNA. In embodiments, the tracrRNA and the crRNA may be separate molecules.

The inverted repeat sequence is configured so that it can concurrently be hybridized to a sequence in a double stranded DNA molecule and to the complementary strand of the DNA, when in the presence of the DNA and a Cas protein. The term “double stranded” DNA as used herein a DNA bubble.

The disclosure includes all complexes that comprise the gRNA, a Cas protein, and DNA as described in the Examples and illustrated in the figures. The disclosure includes such complexes in a cell-free environment, in associate with viral DNA, in individual cells, including prokaryotic and eukaryotic cells in culture, and within cells in multi-cellular organisms, including but not necessarily limited to fungi, plants, and animals. The described compositions and methods may be used, and delivered to cells if desired, for research, diagnostic, prophylactic, and therapeutic purposes.

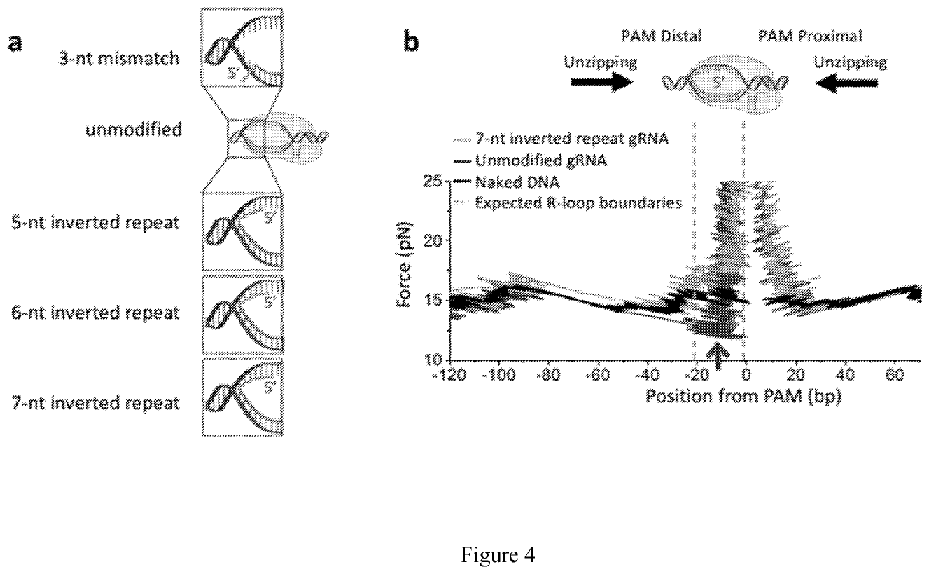

The gRNAs of this disclosure include inverted repeat sequences as appended nucleotides at their at a 5′ or 3′ end ends. An inverted repeat comprises a segment of a gRNA targeted to a spacer sequence in the first strand (canonical Cas effector target strand) of DNA in a double stranded DNA molecule, and a sequence that is targeted to a sequence in the complementary, e.g., a second strand of the double stranded DNA. The inverted repeat sequence is thus configured so that it can concurrently be hybridized to the spacer sequence and to the complementary strand of the DNA when in the presence of the DNA and the Cas protein. A non-limiting illustration of this configuration is shown in FIG. 4, panel b, wherein the appended nucleotides are shown at the 5′ end of the gRNA.

Embodiments of the disclosure are illustrated using dCas9 and dCas12a. The amino sequence of both of these proteins are known in the art. The demonstration using nuclease dead Cas proteins is expected to be extendable to nuclease active Cas proteins that recognize a DNA target in a gRNA-directed manner. Thus, the disclosure is expected to be suitable for use with any Cas enzyme that is a class I or class II CRISPR enzyme, including all types of Cas proteins encompassed by class I and class II CRISPR systems, including but not limited to Cas3/cascade, Cas9, Cas12 and Cas14 systems.

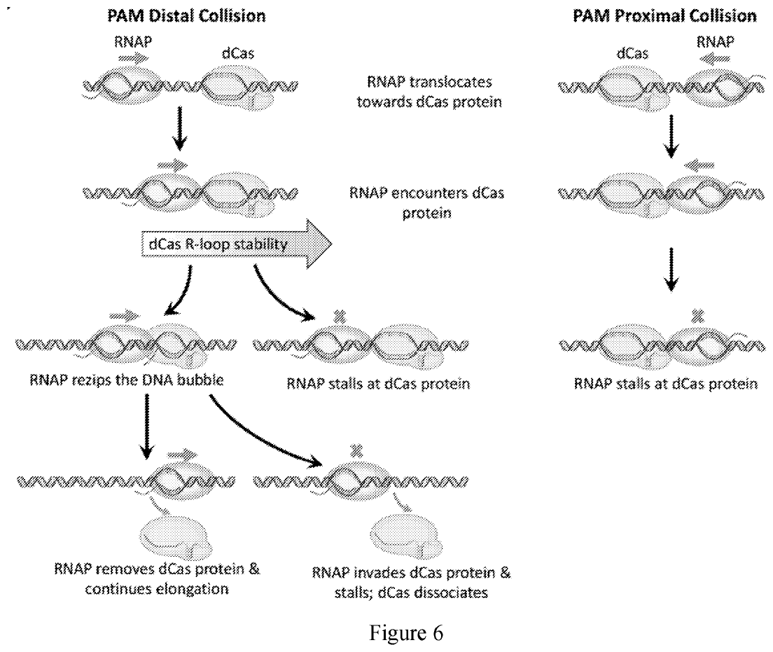

Certain aspects of the disclosure are illustrated using regions that are proximal and distal to the PAM sequence. The meaning of “proximal” and “distal” PAM will be evident to those skilled in the art from the Examples and Figures, such as those illustrating a PAM distal and PAM collision with an RNAP, such as in FIG. 6. In embodiments, the PAM distal side is on the 5′ side of the spacer when the Cas protein is a Ca9, and on the 3′ end for Cas12a.

The disclosure includes using the described Cas proteins and gRNAs for any purpose, non-limiting examples of which include increasing stability of the R-loop, increasing the dwell time of a Cas protein on a DNA substrate, impeding translocation of a motor protein along DNA, and enhancing gene editing, such as by enhancing DNA cleavage, DNA transposition, insertion of a repair template by recombination, correction of single nucleotide mutations or indels, and degradation of DNA, such as by a Cas3 protein.

In one embodiment, the disclosure comprises determining a DNA spacer sequence for targeting with a described modified gRNA in one of more cells, optionally determining a PAM that is linked to the spacer sequence, designing a modified gRNA comprising an inverted repeat sequence that is capable of concurrently hybridizing to the spacer sequence and to the complementary strand of the DNA comprising the spacer sequence, and delivering the modified gRNA and a Cas protein to the cell, whereby the Cas protein binds to a sequence of the DNA comprising the spacer sequence, and wherein one or more properties of the bound Cas protein are different relative to the properties of the same Cas protein targeting the same spacer, but used with an unmodified gRNA. In embodiments, the DNA spacer sequence is unique to a set of cells within a population of cells. As such, the described modified gRNA and Cas protein can selectively target only a subset of the cells with a larger cell population. Larger populations can include but are not necessarily limited to mixed bacteria populations, and normal and abnormal cells, such as normal cells and cancer cells.

Methods for delivering the described Cas proteins and gRNAs to cells, whether in vitro or in vivo can be adapted from known CRISPR delivery systems. In embodiments, the Cas protein and/or the gRNA can be delivered as mRNA or DNA polynucleotides that encode Cas protein and/or the gRNA. It is considered that administering a DNA or RNA encoding any component described herein is also a method of delivering a component to an individual or one or more cells.

Methods of delivering DNA and RNAs encoding proteins and gRNAs are known in the art and can be adapted to deliver the described Cas protein and gRNAs, given the benefit of the present disclosure. In embodiments, one or more expression vectors are used and comprise viral vectors. Thus, in embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus. In embodiments, a recombinant adeno-associated virus (AAV) vector may be used. In certain embodiments, the expression vector is a self-complementary adeno-associated virus (scAAV). Expression vectors encoding the described modified gRNAs are included in the disclosure, as are cDNAs that correspond to the modified gRNAs. In embodiments, the described Cas protein and the gRNA is introduced into a cell in the form of a ribonucleoprotein.

In embodiments, an effective amount of a gRNA and a Cas protein is administered to cells or an individual in need thereof. An effective amount can be determined by those skilled in the art when taking account the rationale for selecting the target sequence, and a disease or disorder or other characteristic that is correlated with the presence of the target sequence.

In embodiments, the modified gRNA may comprise modified nucleotides to, for example, provide resistance to RNA nucleases. In embodiments, the Cas protein may be modified. For example, for use with eukaryotes, the Cas protein may be modified to comprise a nuclear localization signal. In embodiments, the modified gRNA may be used in conjunction with an endogenously expressed Cas protein. In embodiments, the Cas protein may be provided as a component of a fusion protein. The fusion protein may enhance one or more properties of a described CRISPR system, such as improving bioavailability, increasing half-life, enhancing DNA editing, enhancing dwell time of the Cas enzyme on the DNA, and the like.

The following Examples are intended to illustrate but not limit the disclosure.

### Example 1

R-Loop of a dCas Complex Bound to DNA

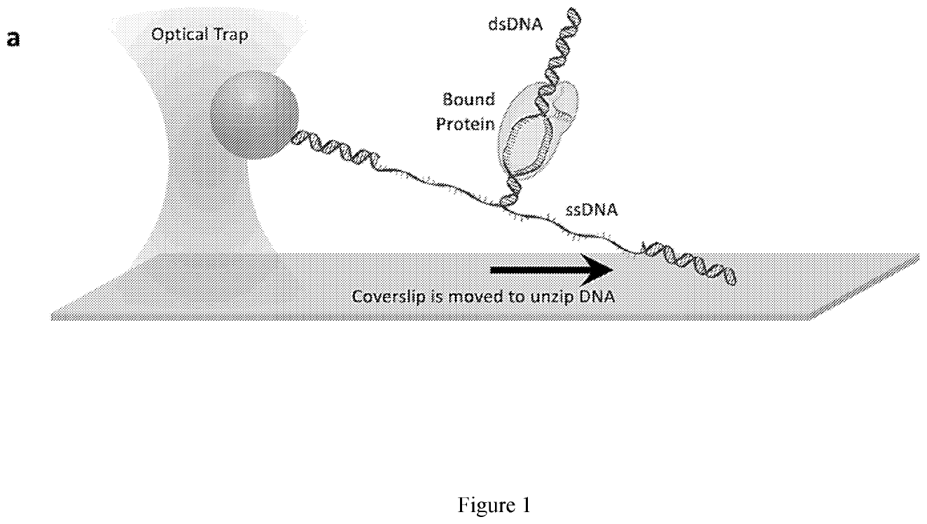

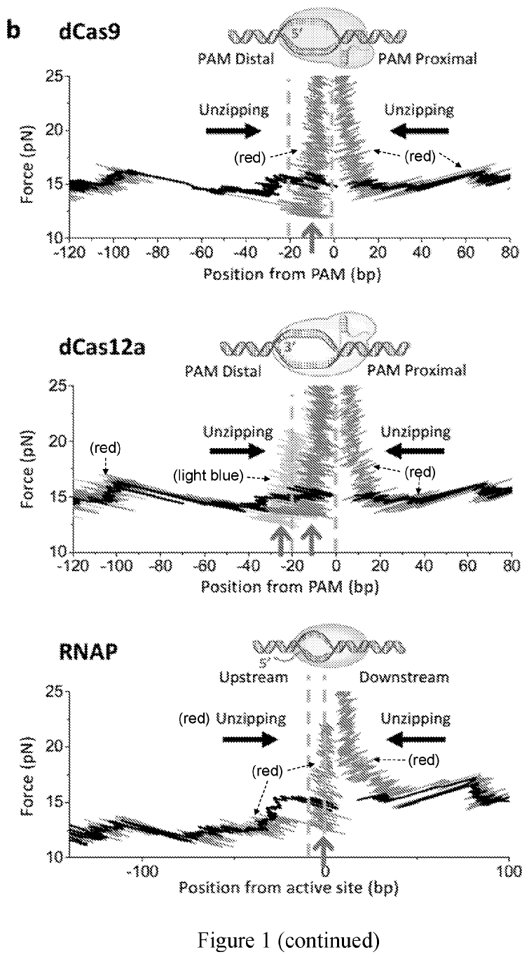

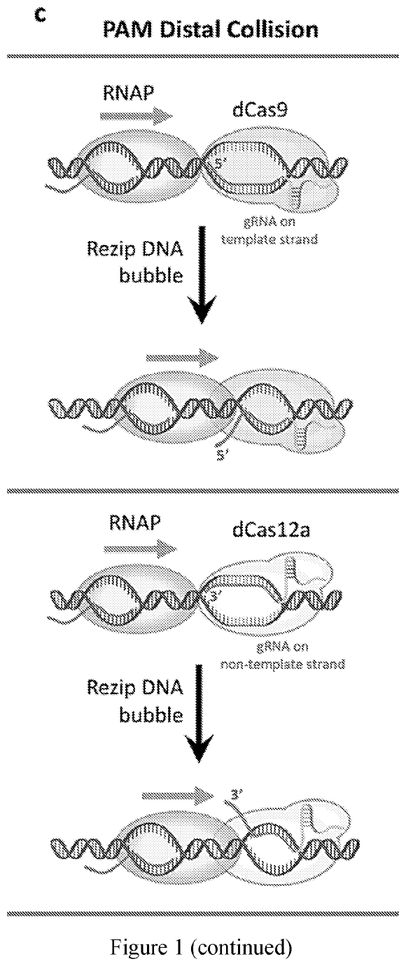

To investigate the structural features that may underlie the polar barrier of a bound dCas, we first mapped protein-nucleic acid interactions of a bound dCas via a high-resolution ‘DNA unzipping mapper’ technique¹¹⁻¹⁴ (FIG. 1a). Using an optical trap, we unzipped DNA by mechanical separation of the two strands of a double-stranded DNA (dsDNA) containing a bound dCas. Before the unzipping fork encountered the bound dCas, the unzipping force followed the force signature of the corresponding naked-DNA baseline; but when the unzipping fork encountered the complex, the unzipping force deviated from the naked DNA baseline, indicating DNA interactions with dCas. To examine interaction polarity, we unzipped the DNA through a bound complex from either the PAM-distal side or the PAM-proximal side.

Using the unzipping mapper, we compared the force signatures of both a dCas9 and dCas12a, two of the most prevalent Cas proteins (FIG. 1b). Although the target sequence of gRNA is located at the 5′-end for dCas9 and at the 3′-end for dCas12a, we found that their interaction maps were strikingly similar.

When unzipped from the PAM-distal side, both dCas complexes showed a force drop below the naked DNA baseline followed by a force rise above the baseline. The force drop is consistent with the presence of the gRNA/DNA hybrid, which prevents DNA base pairing, creates a DNA bubble, and thus reduces the unzipping force. Note that due to thermal DNA “breathing” fluctuations, the unzipping fork detects the DNA bubble downstream¹⁵, leading to an earlier force drop. The force drop indicates a lack of strong interactions between the dCas protein and DNA prior to the bubble. For dCas9, the subsequent force rise was detected within the gRNA/DNA hybrid region, indicating strong interactions between dCas9 and DNA in that region. For dCas12a, two types of traces were detected (middle panel of FIG. 1b), 43% of the 37 traces measured show a single force rise above the naked DNA force baseline within the gRNA/DNA hybrid region, and the remaining traces show an additional force rise above the naked DNA force baseline at the distal end of the gRNA/DNA hybrid. Both types of traces show a force dip below the DNA baseline within the gRNA/DNA hybrid. These observations indicate that while dCas9 assumes one dominant conformation, dCas12a may adopt two distinct conformations, as have been suggested by previous biochemical studies¹⁶⁻²⁰. In contrast, when either a dCas9 or dCas12a was unzipped from the PAM-proximal side, the force rose sharply at ˜ 6 bp from the PAM site, indicating tight binding of the dCas protein to DNA at that region. The locations of this tight binding site are consistent with those suggested by structures of these complexes²¹⁻²³.

Interestingly, these force features bear a remarkable resemblance to those of an E. coli transcription elongation complex (TEC), which the DNA unzipping mapper method previously characterized²⁴⁻²⁶ For ease of direct comparison of data with dCas complexes, we re-mapped the TEC under the same experimental conditions as for the dCas complexes (FIG. 1b). When unzipped from upstream of transcription, a TEC showed a force drop due to the transcription bubble containing the RNA/DNA hybrid, followed by a force rise near the active site. When unzipped from downstream of transcription, a TEC showed a force rise at 10-20 bp downstream of the active site, indicating tight binding of RNAP to DNA downstream of its active site.

### Example 2

Mechanism of dCas Roadblock Polarity

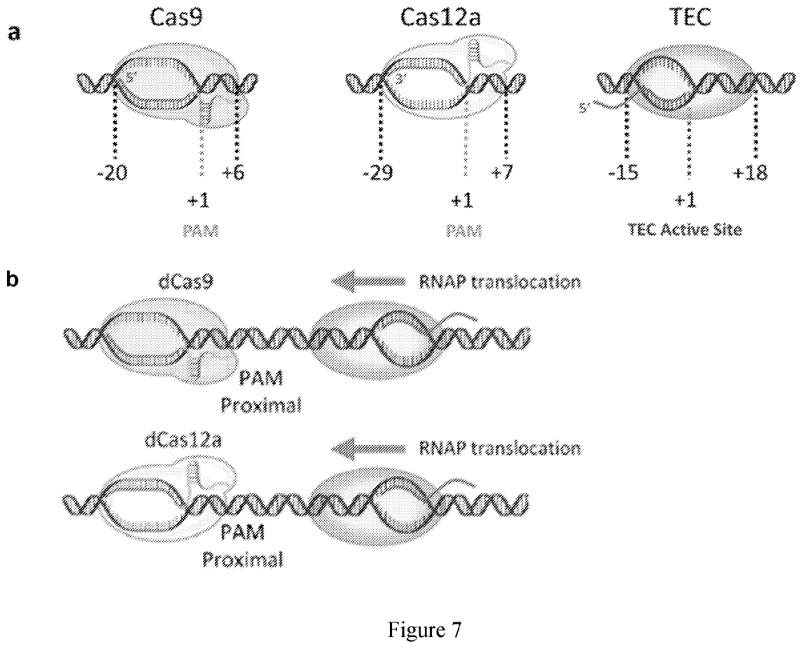

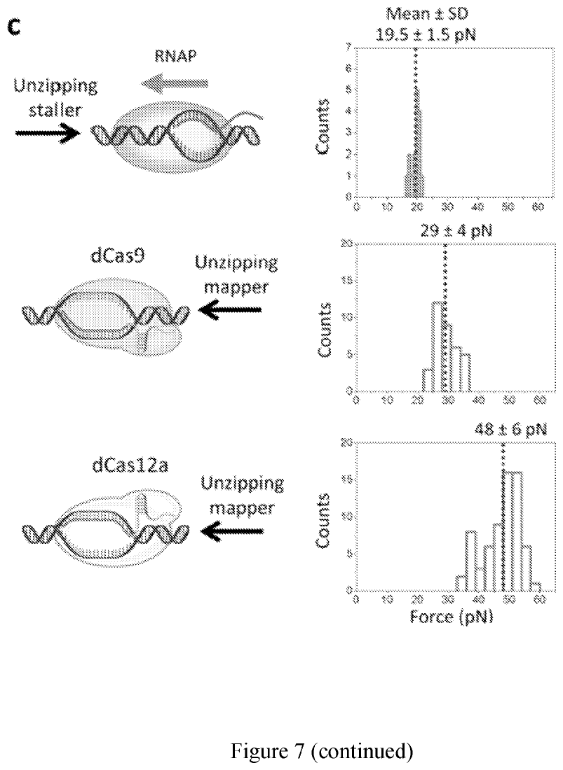

The unzipping mapper data (FIG. 1b) clearly demonstrate that, like a TEC, a DNA-bound dCas complex contains an unprotected R-loop mediated DNA bubble near one end and tightly clamped DNA at the other end. These shared structural features suggest that these complexes may be removed by a common mechanism. Previous bulk transcription studies showed that collapse of the transcription bubble leads to the destabilization of a TEC²⁷⁻²⁹ Thus, without intending to be bound by any particular theory, we considered that a DNA-bound dCas may be destabilized similarly via DNA bubble collapse of a bound dCas. This led to the following non-limiting description of a mechanism for the polarity of CRISPRi. It is considered that this polarity is inherent to the common structural features of TEC and dCas complexes. When a translocating RNAP approaches a bound dCas from the PAM-distal side (FIG. 1c), RNAP first encounters the DNA bubble of the dCas complex. Because RNAP tightly clamps its downstream DNA, forward translocation will rezip the DNA bubble of the dCas complex. This leads to collapse of the DNA bubble of the dCas complex, disruption of the gRNA/DNA hybrid, and ultimately dCas removal from DNA. Thus, transcription from the PAM-distal side is likely to be more permissive. On the other hand, when RNAP approaches a bound dCas from the PAM-proximal side, RNAP will encounter a dCas roadblock that may be too strong for RNAP to overcome (FIG. 7). Thus, transcription from the PAM-proximal side is more prohibitive.

### Example 3

A Bound dCas is a Highly Asymmetric Roadblock

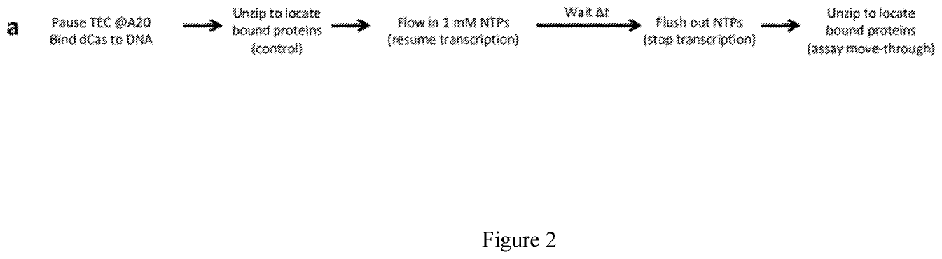

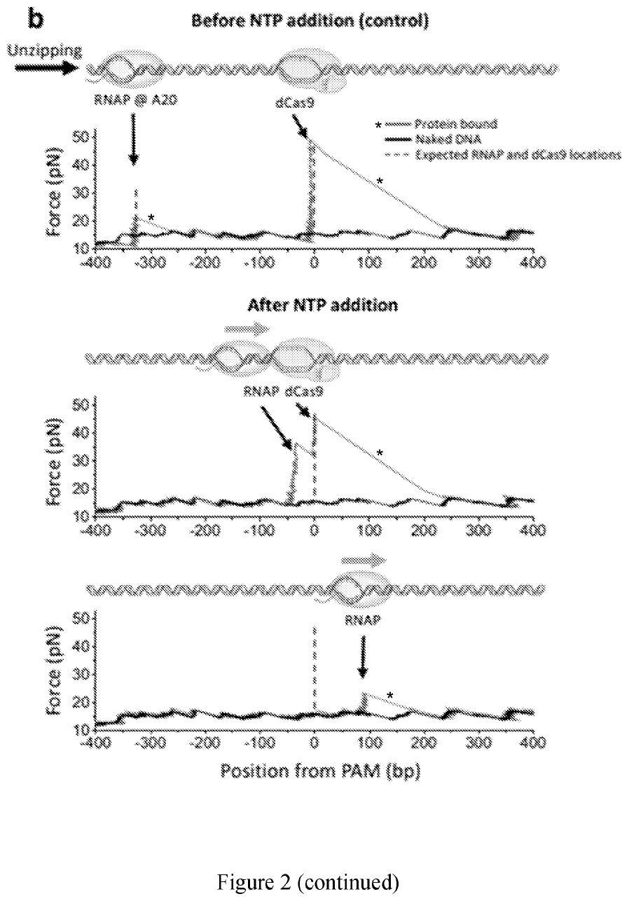

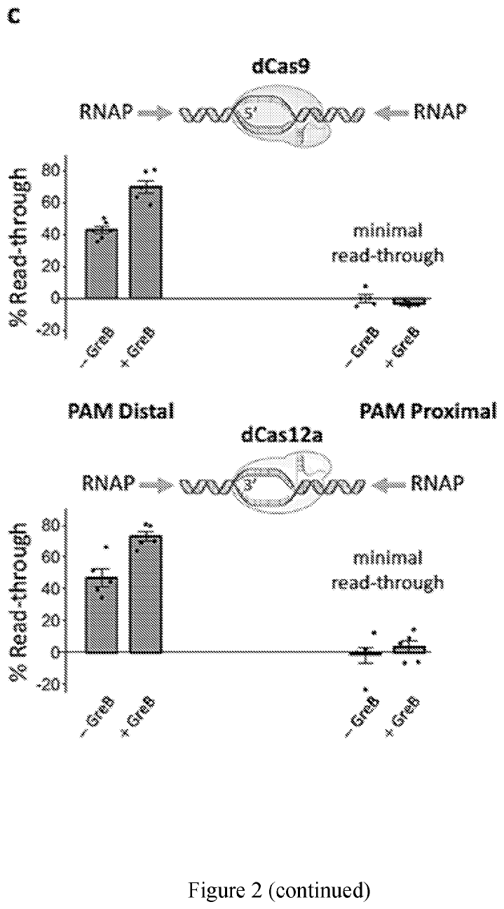

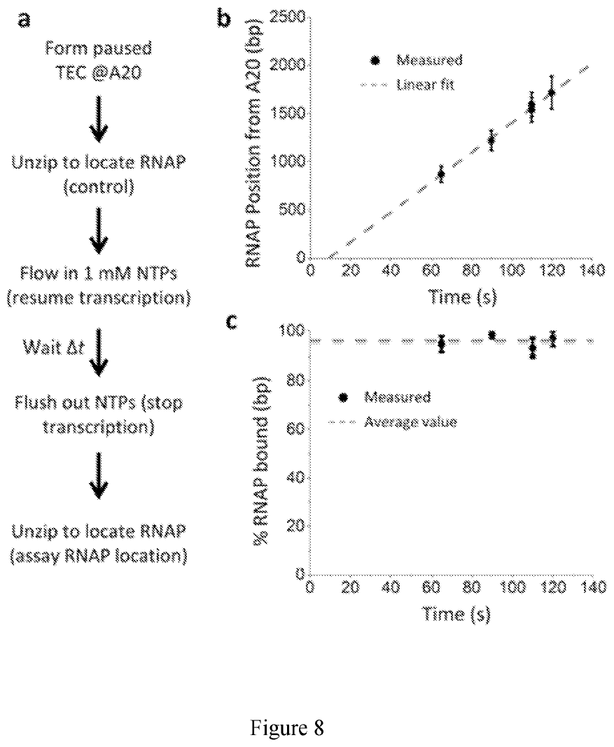

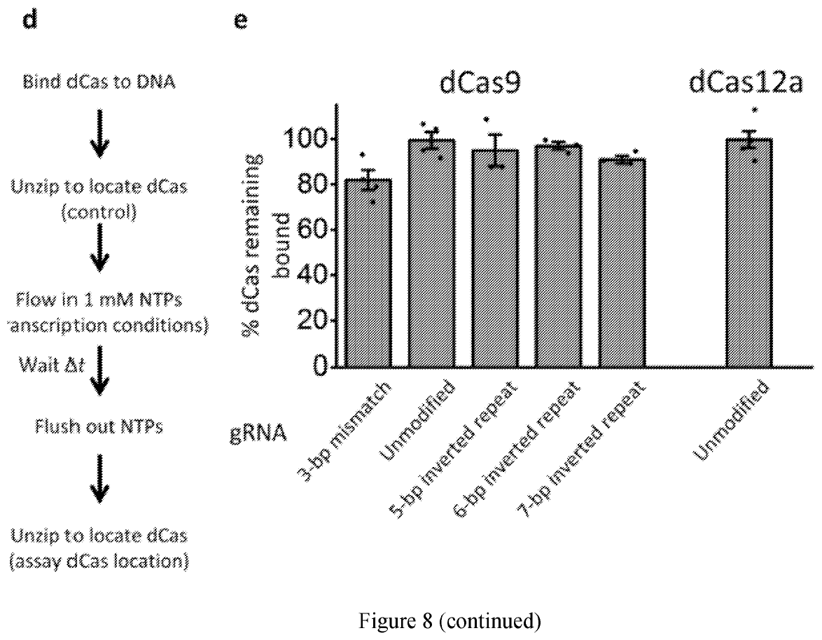

We developed a single-molecule assay using the DNA unzipping mapper that quantitatively measures the ability of RNAP to transcribe through a bound dCas from either the PAM-distal side or PAM-proximal side. In this assay (FIG. 2a), a DNA template initially contained a TEC paused at the A20 position via nucleotide starvation and a bound dCas downstream. A control experiment was conducted using the unzipping mapper to determine the occupancies of RNAP and dCas, which were both found to be >90%. Subsequently, transcription was resumed by the introduction of NTPs into the sample chamber and was then quenched after 135 s, which should have been sufficient time to allow a majority of RNAPs to reach the bound dCas while limiting spontaneous dCas9 dissociation (FIG. 8). Subsequently, the locations of bound proteins were detected using the unzipping mapper.

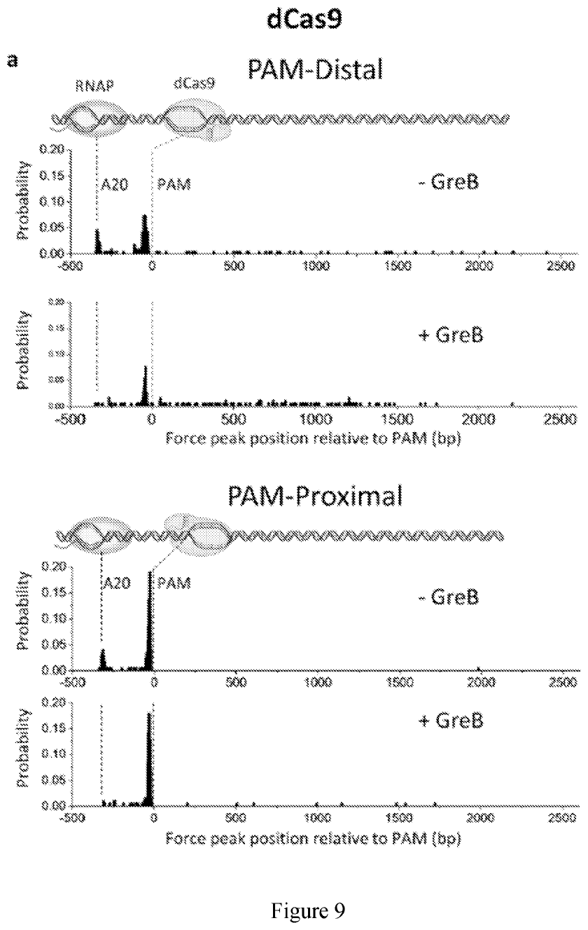

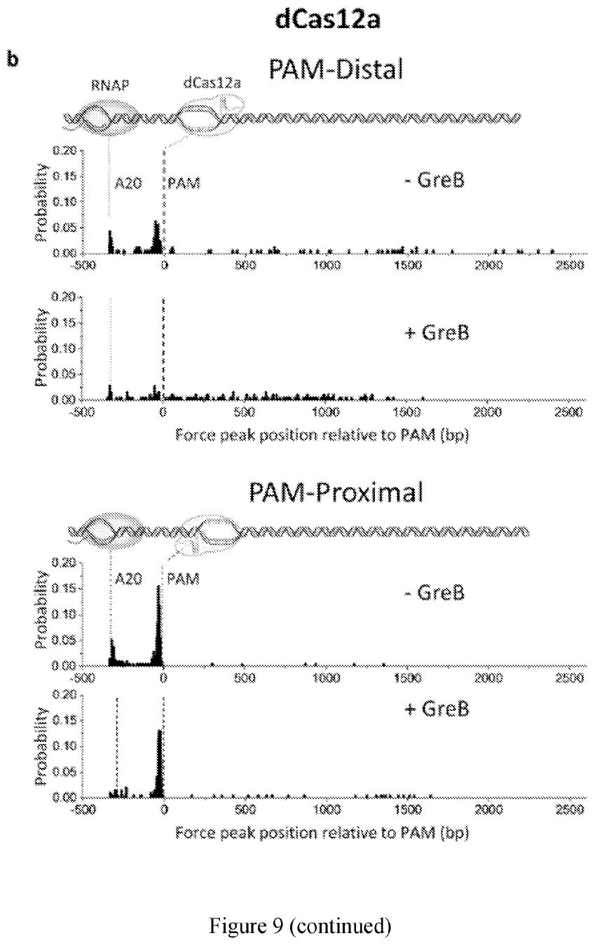

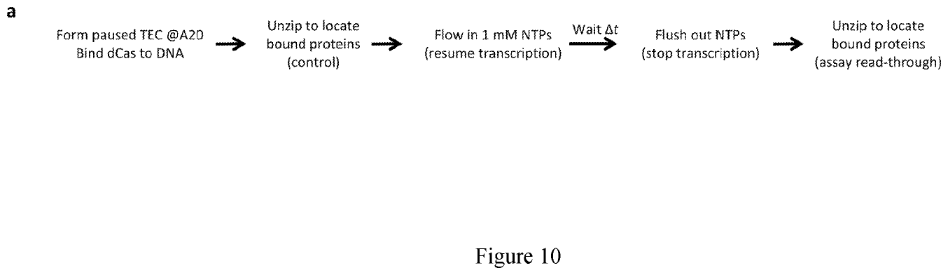

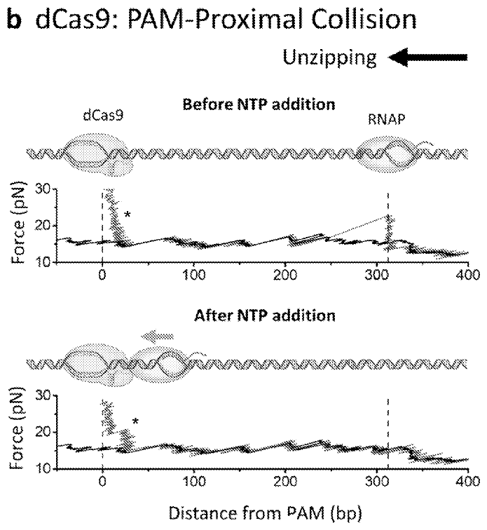

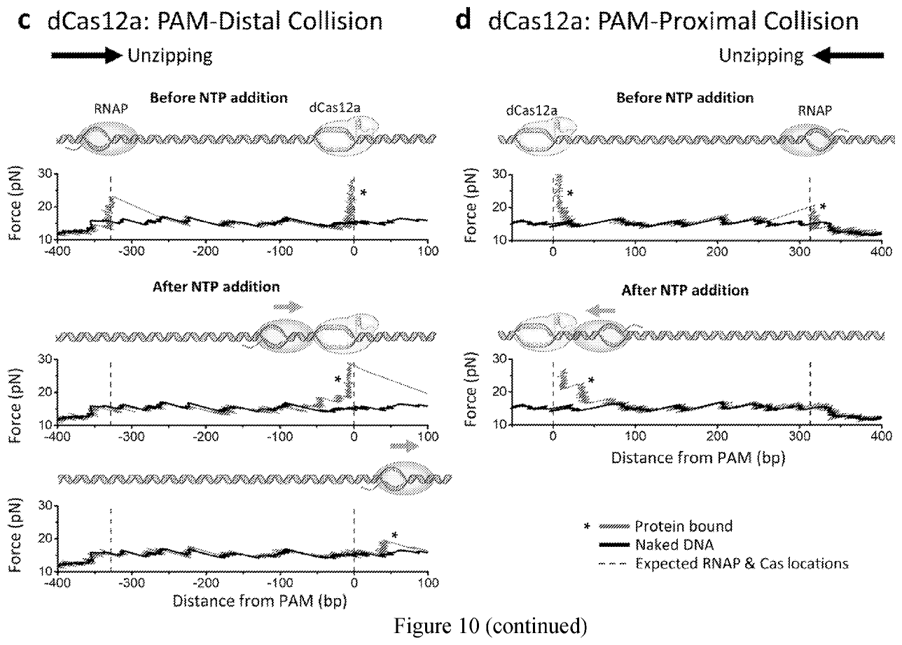

Unzipping traces taken after the NTP chase fell into several categories due to asynchronization of the RNAP population as a result of the stochastic nature of RNAP motion (FIG. 9). FIG. 2b shows representative traces of this assay when transcription approached dCas9 from the PAM-distal side. One example shows a force peak was detected immediately before the dCas9 force peak, suggesting that RNAP was stalled after colliding with dCas9 but was unable to remove dCas9. Another example trace shows that the only detected bound protein was downstream of dCas9, possibly due to RNAP having elongated forward after removing dCas9 but not having reached the template end. In contrast, when RNAP encountered dCas9 from the PAM-proximal end, the majority of traces showed two force rises at around 30 bp and 6 bp before the PAM site (FIGS. 9 and 10b), consistent with RNAP being stalled after colliding with dCas9 but unable to remove dCas9. We also carried out a similar experiment to examine transcription through dCas 12a (FIGS. 9, 10c, and 10d) and obtained a similar result.

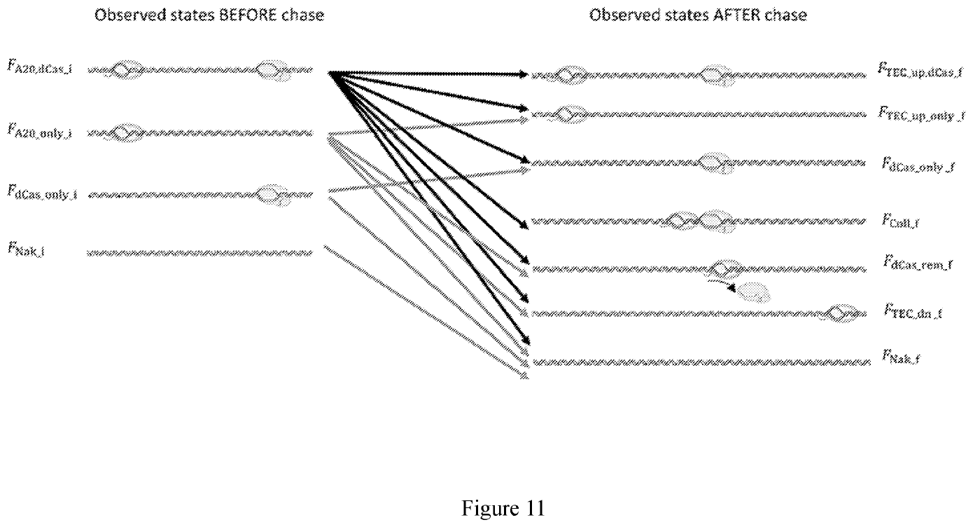

These traces show very different transcription behaviors between the PAM-distal and PAM-proximal collisions and demonstrate that a bound dCas is a polar barrier to transcription. To accurately determine transcription read-though from each side of a bound dCas, we carried out several control experiments to obtain the probability of a template initially not having a bound RNAP or dCas protein (Supplementary Table 1) and the probabilities of spontaneous dissociation of RNAP or dCas (FIGS. 8c and 8e). These probabilities were taken into account in the final read-through analysis (FIG. 11).

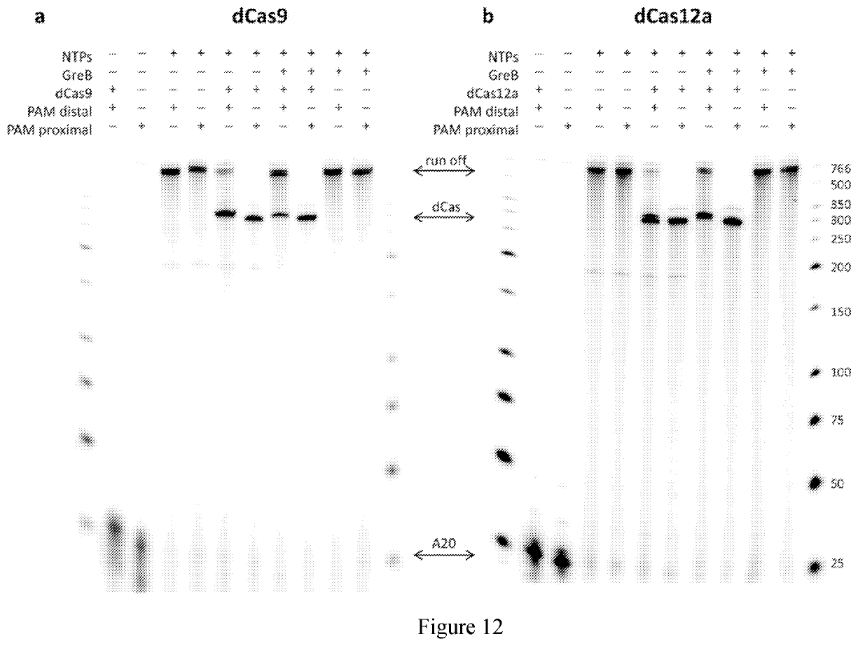

Using this method, we found that transcription read-through of a bound dCas9 showed an efficiency of 43% when RNAP approached dCas9 from the PAM-distal side and was undetectable from the PAM-proximal side (FIG. 2c). For dCas12a, the read-through efficiencies were similar to those for dCas9: 47% when RNAP approached dCas12a from the PAM-distal side and undetectable from the PAM-proximal side (FIG. 2c). To further validate these results from single-molecule studies, we carried out corresponding bulk transcription assays, and the bulk data show a similar extent of polarity (FIG. 12). These findings on the polarity of both the dCas9 and dCas 12a barriers to transcription are consistent with those from previous in vivo studies^{6-8,30,31}, while also providing a highly quantitative and controlled measure of the polarity.

When RNAP encountered a bound dCas but could not read through it, RNAP likely backtracked^{24,32-34}., where RNAP reverse translocates along DNA with its catalytic site disengaged from the 3′-end of the RNA, rendering transcription inactive^{35,36}. E. coli GreB is a transcription elongation factor that is known to rescue backtracked complexes³⁷⁻³⁹. GreB can stimulate the intrinsic cleavage activities of RNAP, leading to the removal of the 3′-end of the RNA and alignment of the newly generated RNA 3′-end with the catalytic site, reactivating transcription. We thus conducted transcription assays in the presence of 1 μM GreB. When RNAP encountered dCas from the PAM-distal side, the transcription read-through efficiency increased significantly, from 43% to 70% for dCas9 and from 47% to 73% for dCas12a. Interestingly, when RNAP encountered dCas from the PAM-proximal side, the read-through efficiency remained essentially zero for both dCas9 and dCas12a. Our bulk transcription assays show a similar effect of GreB on the polarity of transcription read-through (FIG. 12).

This shows that backtracking was likely the main cause of RNAP stalling at a dCas roadblock from the PAM-distal side. While transcription through a bound dCas from the PAM-distal side is facilitated by GreB, transcription through dCas from the PAM-proximal side encounters a nearly insurmountable obstacle and cannot be rescued by GreB. Thus, in the presence of GreB, a bound dCas becomes an even more highly asymmetric and polar barrier to transcription. This ultimately results from a bound dCas complex having an unprotected DNA bubble that can be rezipped and collapsed by RNAP.

### Example 4

### A DNA Translocase Exhibits the Same Polarity

An important prediction of the hypothesized mechanism is that a bound dCas should be a polar barrier not just to RNAP, but to any DNA translocase capable of rezipping downstream DNA. To test this possibility, we required a translocase to approach a bound dCas from a defined direction. E. coli Mfd met this requirement as it interacts with a TEC stalled at a defined location, making it possible to control the position and orientation of translocation^{26,40-42}. In the presence of ATP, Mfd can bind to the stalled TEC and forward translocate to disrupt the TEC, before processively continuing translocation in the same direction as the disrupted TEC.

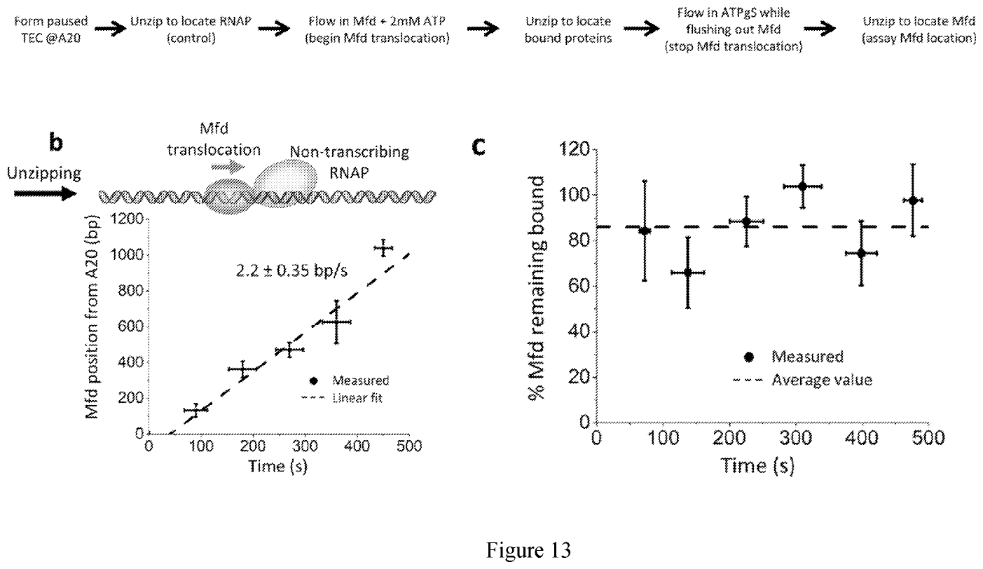

Using this method of loading Mfd onto DNA, we found that in ˜ 85% of traces that initially contained a TEC, Mfd remained associated with DNA and translocated processively along DNA over a long distance at a rate of 2.2 bp/s (FIG. 13). The non-transcribing RNAP was presumably associated with Mfd^{26,43-45}. This control experiment demonstrates that Mfd can serve as a translocase and approach a bound dCas with the start and end of the translocation under the control of the ATP chase and quench.

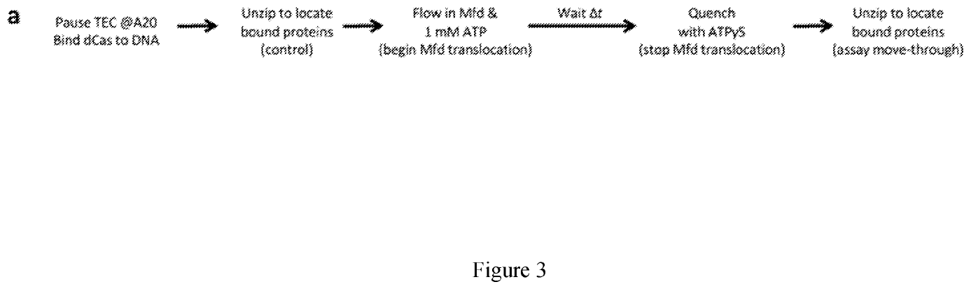

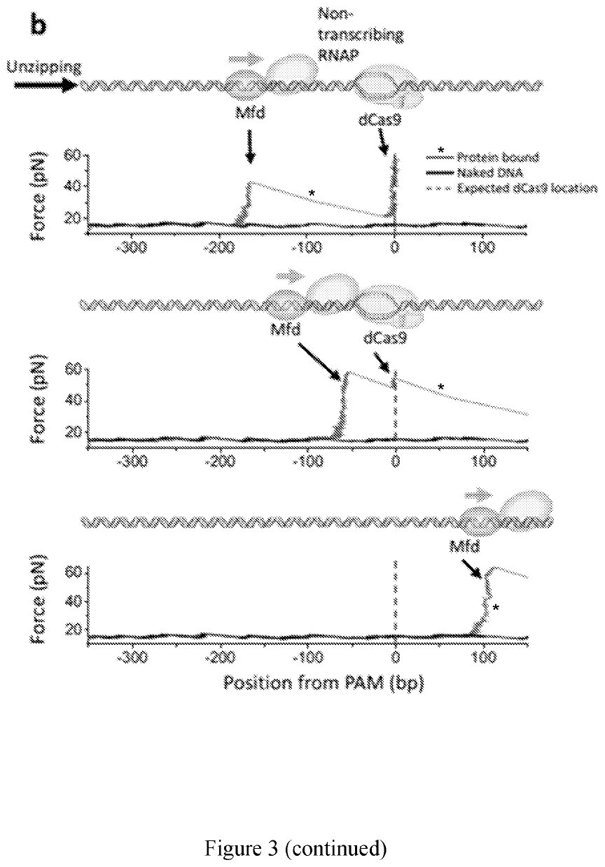

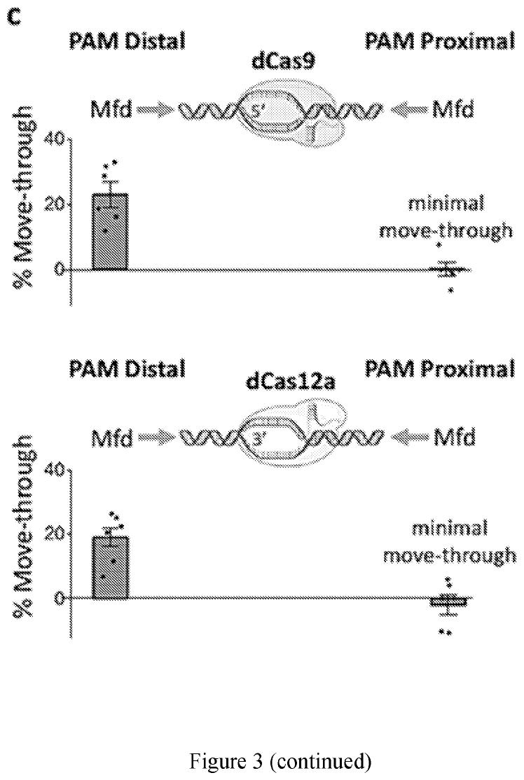

To examine whether Mfd experiences a bound dCas as a polar barrier, we performed experiments similar to those presented in FIG. 2, except with an active Mfd instead of RNAP (FIG. 3a). Since Mfd translocation is substantially slower than RNAP translocation (compare FIG. 8b and FIG. 13b), Mfd was allowed to translocate for 480 s, so that most Mfd should reach a bound dCas before the reaction was quenched. The outcomes of Mfd collision with dCas9 were then assayed using the unzipping mapper.

FIG. 3b shows example traces of Mfd approaching dCas9 from the PAM-distal side. One example trace shows a force peak detected prior to dCas9, consistent with Mfd not having reached the bound dCas. A second example trace shows a force peak detected immediately prior to a bound dCas9, consistent with Mfd colliding with dCas9. A third example trace shows a bound protein detected downstream of the dCas9 binding site, consistent with Mfd-mediated removal of dCas9 and continued translocation.

We classified the traces into different categories to determine Mfd move-through efficiency when Mfd encountered dCas9 or dCas12a from either the PAM-distal side or the PAM-proximal side (FIG. 3c; FIG. 11). For either dCas, Mfd move-through efficiency was ˜ 20% when encountering the bound dCas from the PAM-distal side and was undetectable when encountering dCas from the PAM-proximal side. Thus, Mfd senses the same polarity as RNAP, providing strong evidence for the described mechanism of the dCas roadblock polarity.

We also noted that when encountering a dCas from the PAM-distal side, Mfd showed a lower move-through efficiency than RNAP. We attribute this to the difference in the stability of two motor proteins when working against a strong roadblock. While RNAP can remain stably bound to the substrate, thus allowing for multiple attempts to overcome the barrier, Mfd may tend to dissociate when working against a strong roadblock²⁶, reducing its opportunity to continue to work against the barrier.

### Example 5

### Modulation of Transcription Roadblock Read-Through

The described data also support strategies to modulate dCas roadblock polarity to transcription For example, transcription read-through from the PAM-distal side of a dCas complex relies on disruption of the R-loop and collapse of the DNA bubble, which depend on gRNA interactions with DNA. Thus, if a gRNA can be modified to increase (decrease) the stability of the R-loop, then transcription read-through may be down-(up-) regulated.

To increase the stability of the R-loop of a bound dCas9, we extended the 5′ end of the original gRNA with an inverted repeat sequence (FIG. 4a). This modified gRNA could form an extended R-loop that straddles across the DNA bubble's two strands. For RNAP to transcribe through dCas9 complex containing this modified gRNA, RNAP must disrupt both the RNA/DNA hybrid on the template strand as well as the RNA/DNA hybrid on the non-template strand. The reinforced resistance by the two RNA/DNA hybrids should make it more difficult for RNAP to rewind and collapse the DNA bubble of the dCas9 complex.

We examined how such a modified gRNA impacted dCas9 binding to DNA by unzipping through the bound dCas9 using the unzipping mapper. FIG. 4b shows one set of example traces of a bound dCas9 containing a gRNA with a 7-nt inverted repeat sequence at the 5′ end. While this modified gRNA resulted in little change in the force signature for unzipping from the PAM-proximal side, the force drop which was observed with the original gRNA for unzipping from the PAM-distal side was no longer present, and the unzipping force at the expected hybrid location was slightly above that of the naked DNA baseline. This observation provides evidence for the presence of an R-loop that bridges the two DNA strands due to the presence of modified gRNA complexed with the bound dCas9. For the unzipping fork to proceed, the RNA/DNA hybrids formed at both DNA strands must be disrupted, elevating the force required for unzipping. Although this gRNA sequence could also form a short RNA hairpin at the 5′ end, about 70% of the unzipping traces did not show a force drop when unzipped from the PAM-distal side as in FIG. 4b, suggesting that the RNA configuration that straddles the DNA fork may be more stable than that with an RNA hairpin.

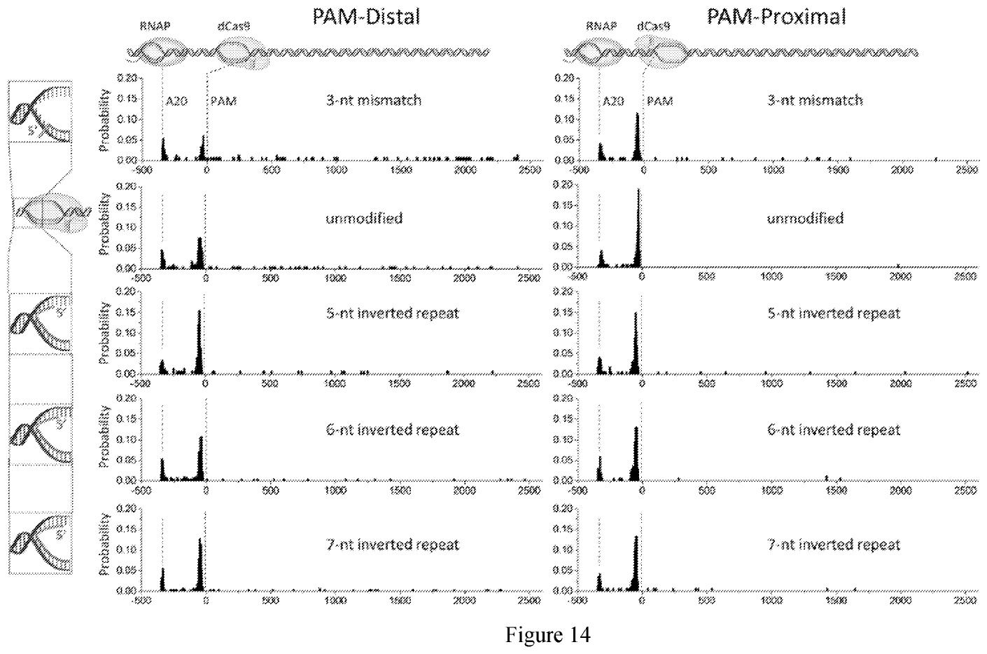

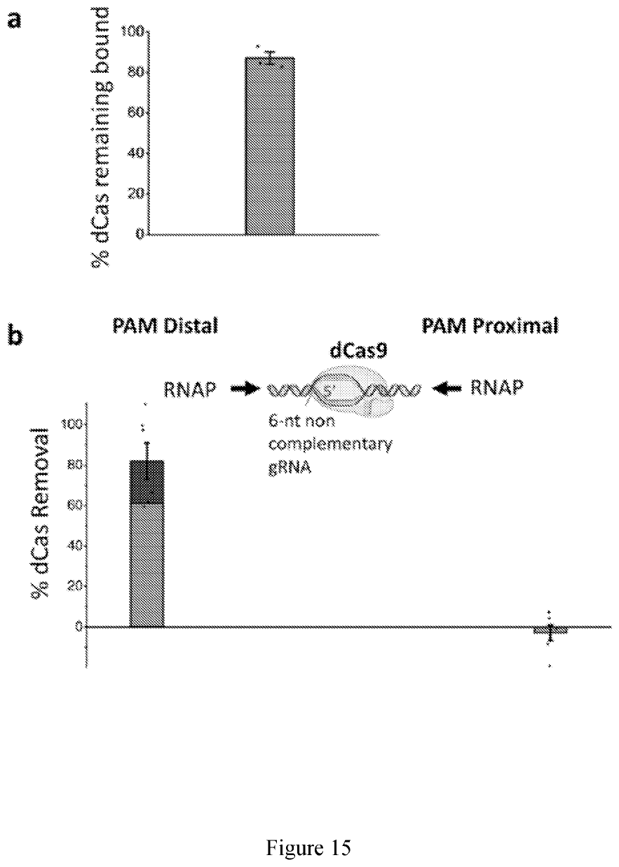

We have also determined how dCas9 containing such a modified gRNA modulates transcription read-through by repeating the assays outlined in FIG. 2 using extended gRNAs containing a 5-nt, 6-nt, or 7-nt inverted repeat (FIG. 4c; FIGS. 8e and 14). For all three modified gRNAs, transcription read-through from the PAM-proximal side remained essentially zero, while transcription read-through from the PAM-distal side decreased from 43% to 18%, 10%, and 10% for the 5-nt, 6-nt, and 7 nt-inverted repeat gRNA, respectively. To test if the observed PAM-distal barrier enhancement was merely a result of the extension of the gRNA, we conducted control experiments using a gRNA with an extension that is not complementary to the unmodified gRNA so that the extended sequence cannot hybridize with the DNA bubble of a bound dCas9 or DNA in the vicinity. We detected a significant increase in read-through from the PAM-distal side without any detectable change in read-through from the PAM-proximal side, suggesting that the extended non-complementary sequence hangs away from the R-loop of a bound dCas9 and facilitates the start of RNA-DNA separation during RNAP invasion (FIG. 15). Thus, the barrier enhancement from a modified gRNA with an inverted repeat should not be a result of the mere extension of the gRNA.

To determine whether transcription read-through from the PAM-distal side of a bound dCas9 can also be up-regulated, we introduced a 3-nt mismatch to the gRNA at its 5′-end (FIG. 4c; FIG. 14). This mismatch should weaken the RNA/DNA hybrid, making it easier for RNAP to rezip the DNA in the bubble by disrupting the RNA/DNA hybrid. Indeed, we found that transcription read-through from the PAM-distal side increased from 43% to 61% with the 3-nt mismatch gRNA. The increase in the read-through efficiency also reflects a weakened binding of dCas9. Consistent with this, we found that a bound dCas9 containing a 3-nt mismatch showed a much faster spontaneous dissociation rate than a bound dCas9 containing an unmodified gRNA (Supplementary Table 2; FIG. 8e).

Collectively, these results clearly show that transcription read-through from the PAM-distal side of dCas9 can be considerably impacted via gRNA modifications. This finding also serves as strong evidence for R-loop disruption and DNA bubble collapse as a mechanism of transcription read-through.

### Example 6

### Modulation of Transcription Roadblock Removal

The preceding examples characterized the polarity of the dCas roadblock to transcription read-though, which requires the removal of the roadblock by RNAP, followed by transcription through the dCas binding site. An alternative characterization of the roadblock polarity is the efficiency of transcription roadblock removal, which requires the removal of the roadblock by RNAP but does not require RNAP to read through the dCas binding site.

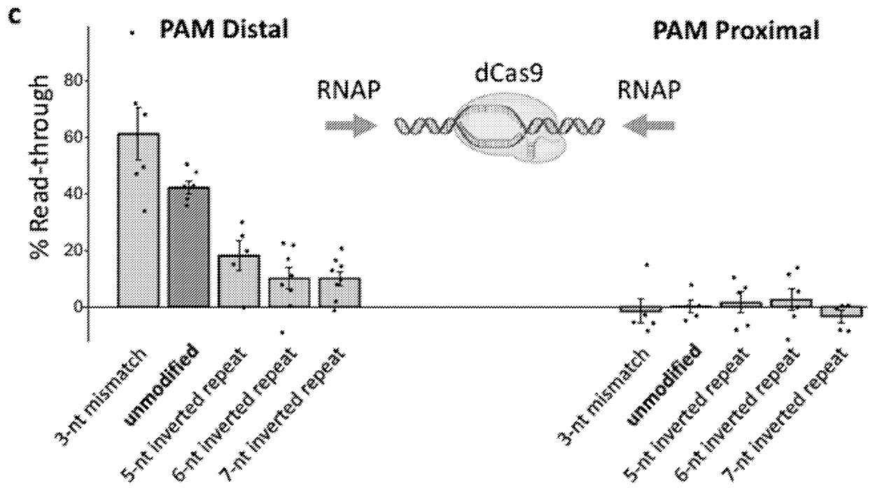

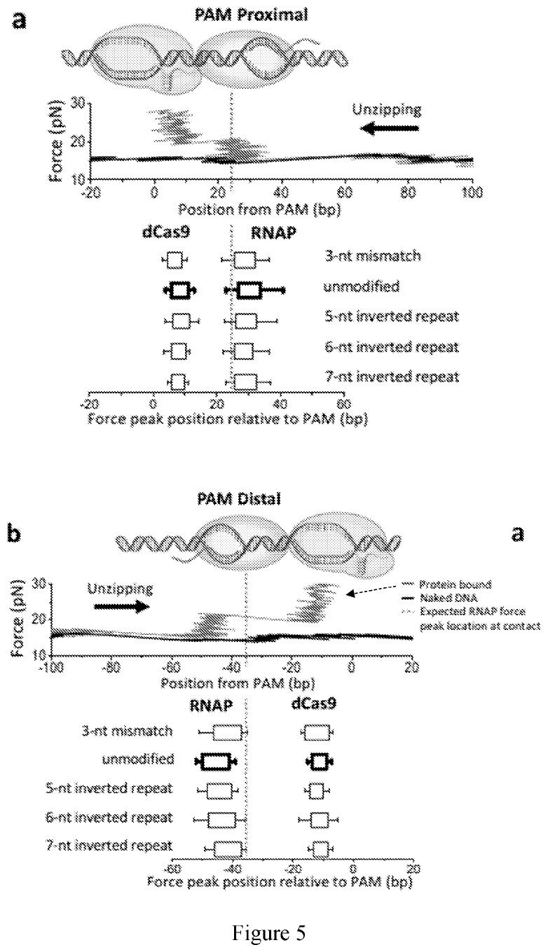

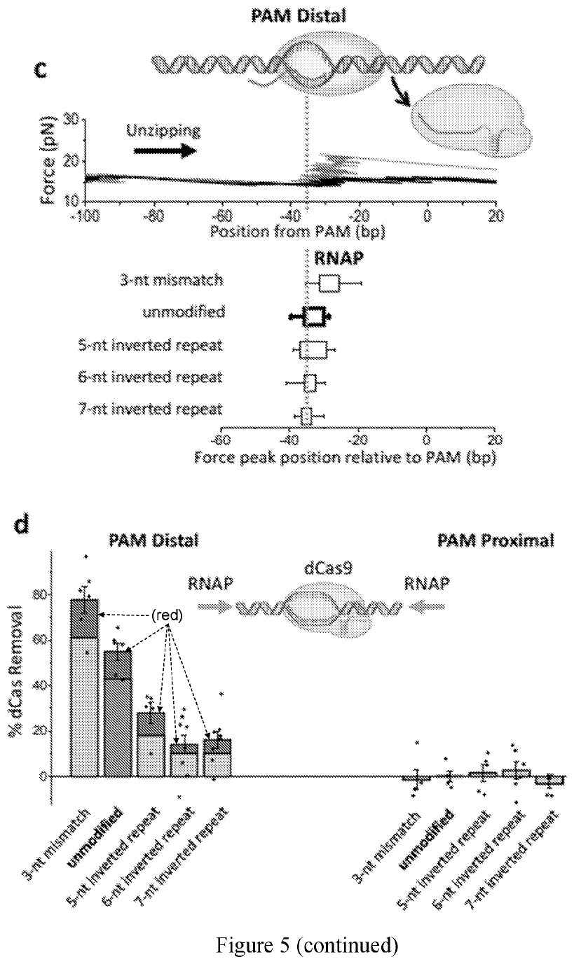

Roadblock removal includes transcription read-through and an additional scenario where RNAP collided with and removed the dCas, but then became stalled. To examine this, we focused on transcription data with an RNAP force signature near the expected dCas9 binding site, corresponding to stalled RNAP after collision with dCas9 (FIG. 5). Note that these traces did not result in read-through.

For PAM-proximal collisions, all traces showed both a bound RNAP and dCas9 (FIG. 5a), indicating that although RNAP collided with dCas9, RNAP could not remove it. Traces in this category showed a force signature consistent with the footprint of RNAP having no overlap with that of dCas9 (FIG. 7a). The spread in the RNAP position may be a result of RNAP backtracking after collision with dCas9.

For PAM-distal collisions, the traces fall into two distinct categories. Just as with the PAM-proximal collisions, one category of traces shows both a bound RNAP and dCas9 (FIG. 5b), consistent with the footprint of RNAP having no overlap with that of dCas9. However, the other category of traces shows only a bound RNAP, indicating that after RNAP collided with dCas9, RNAP removed dCas9, but was then stalled in the process (FIG. 5c). In these traces, the footprint of RNAP showed a significant overlap with the expected dCas9 footprint, indicating significant invasion of RNAP into the dCas complex which was subsequently dissociated. Interestingly, the distance of this invasion decreases with an increase in the stability of the R-loop of dCas9. For example, for a dCas9 complex with a 3-nt mismatched gRNA, the invasion was about 7 nt into the R-loop of dCas9; whereas for dCas9 complex with a 7-nt inverted repeat gRNA, there is minimal invasion. These data show that the weaker the R-loop of dCas9, the easier it is for RNAP to invade and rezip the DNA bubble of dCas9, ultimately removing dCas9. Consistent with this, the fraction of collision traces with dCas9 removed also decreased with an increase in the R-loop stability of dCas9 (FIG. 5d).

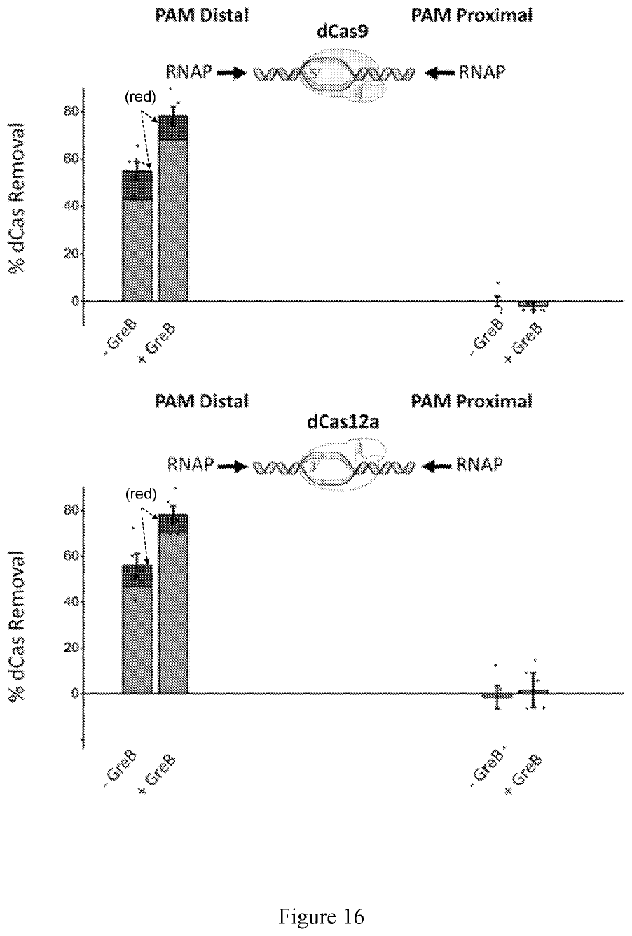

The overall roadblock removal efficiency, considering both the collision traces and read-through traces, shows that dCas9 removal is also polar (FIG. 5d). In comparison with the read-through efficiency (FIG. 4c), the roadblock removal efficiency shows an even greater polarity, and this polarity can also be modulated via modification of the gRNA (FIG. 5d) and transcription factors, such as GreB (FIG. 16).

### Discussion of Examples

Using the CRISPRi system, this disclosure presents high-resolution structural features of dCas-DNA interactions, elucidates the nature of dCas removal by motor proteins, and details the highly tunable nature of dCas removal through modifications of the gRNA.

The disclosure provides a mechanistic explanation for the roadblock polarity that dCas presents to transcription in CRISPRi (FIG. 6). When approaching a bound dCas from the PAM-distal site, RNAP may be able to remove the dCas by disrupting the R-loop, rezipping the DNA bubble, and removing the dCas. In contrast, when approaching a bound dCas from the PAM-proximal site, the R-loop is inaccessible to RNAP, and thus RNAP seems to encounter an insurmountable obstacle. We show that this explanation of the polarity holds for both dCas9 and dCas 12a, which have their target sequences of gRNA located at the 5′-end and the 3′-end, respectively. We also predicted that other dsDNA translocases should sense the same roadblock polarity as RNAP and verified this prediction using Mfd. We further demonstrate that both transcription read-through and roadblock removal by transcription from the PAM-distal side can be modulated by gRNA modifications that alter the R-loop stability of a bound dCas9 complex.

We also show that GreB can significantly facilitate RNAP read-through when RNAP encounters a bound dCas from the PAM-distal side, but has no detectable effect on read-through when RNAP encounters a bound dCas from the PAM-proximal side, demonstrating that dCas is a highly asymmetric and polar barrier to transcription.

In addition to CRISPRi, dCas complexes have also been used to hinder replication. In contrast to transcription, this hindrance was not found to be polar^{9,10}. The described mechanism allows for both the presence of polarity for transcription and the absence of polarity for replication. In order for RNAP to read through a dCas roadblock from the PAM-distal side, RNAP must rezip the DNA downstream to collapse the R-loop of the dCas complex, and thus the ability to rezip is important for read-through. In contrast, a replisome relies on its helicase to unzip DNA to strand separate, and therefore, cannot rezip to collapse the R-loop of a bound dCas complex. To our knowledge, this is the first mechanistic explanation of these apparently disparate findings of dCas roadblock polarity for transcription and replication.

Beyond CRISPRi, dCas proteins are used in a host of other cellular applications. For example, they can be fused to other proteins to direct them to specific loci. The disclosure includes inverted repeat modifications of gRNA sequences to increase the overall stability of dCas9.

Besides engineered dCas proteins, naturally occurring Cas proteins without any inherent nuclease activity are known to direct DNA transposition. In these transposon-associated CRISPR-Cas systems, Cas binding is followed by recruitment of multiple other enzymes that then direct transposition. These systems have been repurposed for gene editing⁴⁸⁻⁵⁰. The stability of bound Cas complexes in these systems is expected to be governed by the same mechanism described in this disclosure, and as such the disclosure includes modulation of this stability to improve the efficiency of transposition and gene editing.

While the present disclosure provides representative embodiments using dCas proteins, the disclosure encompasses other DNA editing proteins. For example, when insertions/deletions are created via non-homologous end joining (NHEJ), gene editing may be enhanced by removal of post-cleavage Cas9 via transcription machinery, which exposes a double-strand break for repair by NHEJ⁵¹. However, this removal may not be desirable if the goal is to utilize homology-directed repair (HDR) to perform precise edits. Cas9 removal may contribute to the observed high probability of the NHEJ pathway selected over the HDR pathway^{51,52}. Cas nuclease removal can also likely be modulated using the same strategy of gRNA modifications as we herein. Thus, the disclosure includes modulation of Cas9 removal to provide improved control over the partition between the HDR and NHEJ pathways.

This disclosure provides in part a mechanistic explanation of dCas roadblock polarity and demonstrates the importance of R-loop stability. Without intending to be bound by any particular theory the disclosure indicates two avenues that impact Cas binding-stability of the R-loop and access to the R-loop. The disclosure includes optimizing and customizing Cas binding using modifications to the gRNA to alter the gRNA/DNA interactions and modulation of protein-DNA interactions to regulate R-loop accessibility. Understanding Cas binding stability also provides a framework to impact the efficiencies of CRISPR applications.

Supplementary Table 1. Trace categories for assaying transcription read-through of a bound dCas protein. This tables shows the detailed trace category classification for RNAP approaching a bound dCas9 complexed with an unmodified RNA from two representative sample chambers, one for PAM-distal and one for PAM-proximal. These fractions (top) are used to compute various probabilities (bottom).

| | | |
|---|---|---|
| Category | dCas9 PAM Distal | dCas9 PAM Proximal |

Before Chase

| | | |
|---|---|---|
| F_{A20, dCas}_{_{—}}ᵢ | 0.91 | 0.93 |
| F_{A20}_{_{—}}_{only}_{_{—}}ᵢ | 0.05 | 0.07 |
| F_{dCas}_{_{—}}_{only}_{_{—}}ᵢ | 0.05 | 0.00 |
| F_{Nak}_{_{—}}ᵢ | 0.00 | 0.00 |
| Total | 1.00 | 1.00 |

After Chase

| | | |
|---|---|---|
| F_{TEC}_{_{—}}_{up, dCas}_{_{—}}_{f} | 0.29 | 0.32 |
| F_{TEC}_{_{—}}ᵤₚ_{_{—}}_{only}_{_{—}}_{f} | 0.03 | 0.05 |
| F_{dCas}_{_{—}}_{only}_{_{—}}_{f} | 0.03 | 0.05 |
| F_{Coll}_{_{—}}_{f} | 0.29 | 0.54 |
| F_{dCas}_{_{—}}ᵣₑₘ_{_{—}}_{f} | 0.06 | 0.00 |
| F_{TEC}_{_{—}}_{dn}_{_{—}}_{f} | 0.26 | 0.02 |
| F_{Nak}_{_{—}}_{f} | 0.03 | 0.02 |
| Total | 1.00 | 1.00 |

Calculated Results

| | | |
|---|---|---|
| P_{Coll}_{_{—}}_{Comp} | 0.64 | 0.61 |
| P_{Read-through} | 0.48 | 0.00 |
| P_{Removal} | 0.58 | 0.00 |

Supplementary Table 2. gRNAs used in this disclosure. Custom Cas9 sgRNAs were purchased from Sigma-Aldrich. Cas12a gRNAs were made by in vitro transcription as described in the Examples. Mismatch and inverted repeat nucleotides are in bold, as indicated.

| gRNA | SEQ ID NO: | RNA sequence (5′ -> 3′) |
|---|---|---|
| Cas9 unmodified gRNA | 1 | GCGCGUAUCAUCCCUUACCG + 80 bp (crRNA + tracrRNA) |
| Cas9 3-nt mismatch | 2 | CGCCGUAUCAUCCCUUACCG + 80 bp (crRNA + tracrRNA) |
| gRNA | 3 | CGCGCUGCGCGUAUCAUCCCUUACCG + 80 bp (crRNA + |
| Cas9 5-nt inverted | 4 | tracrRNA) ACGCGCUGCGCGUAUCAUCCCUUACCG + 80 bp |
| repeat gRNA | | (crRNA + tracrRNA) |
| Cas9 6-nt inverted | 5 | UACGCGCUGCGCGUAUCAUCCCUUACCG + 80 bp (crRNA + |
| repeat gRNA | | tracrRNA) |
| Cas9 7-nt inverted | 6 | AUAUUGGCGCGUAUCAUCCCUUACCG + 80 bp (crRNA + |
| repeat gRNA | | tracrRNA) |
| Cas9 6-nt non | | |
| complementary gRNA | | |
| Cas12a unmodified gRNA | 7 | GGUAAUUUCUACUCUUGUAGAUGCCAUUCCCUACUAUGCGCG |

References for the foregoing text. The reference listings are not an indication that any reference is material to patentability.
1. Jinek, M. et al. A programmable dual-RNA-guided DNA endonuclease in adaptive bacterial immunity. Science 337, 816-821 (2012).
2. Cong, L. et al. Multiplex Genome Engineering Using CRISPR/Cas Systems. Science 339, 819 (2013).
3. Jiang, Y. et al. Multigene Editing in the Escherichia coli Genome via the CRISPR-Cas9 System. (2015).
4. Biot-Pelletier, D. & Martin, V. J. J. Seamless site-directed mutagenesis of the Saccharomyces cerevisiae genome using CRISPR-Cas9. Vol. 10 (BioMed Central Ltd., 2016).
5. Sternberg, S. H., Redding, S., Jinek, M., Greene, E. C. & Doudna, J. A. DNA interrogation by the CRISPR RNA-guided endonuclease Cas9. Nature 507, 62-67 (2014).
6. Qi, L. S. et al. Repurposing CRISPR as an RNA-guided platform for sequence-specific control of gene expression. Cell 152, 1173-1183 (2013).
7. Bikard, D. et al. Programmable repression and activation of bacterial gene expression using an engineered CRISPR-Cas system. Nucleic Acids Research 41, 7429-7437 (2013).
8. Schilling, C., Koffas, M. A. G., Sieber, V. & Schmid, J. Novel Prokaryotic CRISPR-Cas12a-Based Tool for Programmable Transcriptional Activation and Repression. ACS Synthetic Biology 9, 3353-3363 (2021).
9. Schauer, G. D. et al. Replisome bypass of a protein-based R-loop block by Pif1. Proceedings of the National Academy of Sciences 117, 30354-30361 (2020).
10. Whinn, K. S. et al. Nuclease dead Cas9 is a programmable roadblock for DNA replication. Scientific reports 9, 1-9 (2019).
11. Koch, S. J., Shundrovsky, A., Jantzen, B. C. & Wang, M. D. Probing Protein-DNA Interactions by Unzipping a Single DNA Double Helix. Biophysical Journal 83, 1098-1105 (2002).
12. Koch. S. J. & Wang, M. D. Dynamic force spectroscopy of protein-DNA interactions by unzipping DNA. Physical Review Letters 91 (2003).
13. Shundrovsky, A., Smith, C. L., Lis, J. T., Peterson, C. L. & Wang, M. D. Probing SWI/SNF remodeling of the nucleosome by unzipping single DNA molecules. Nature Structural & Molecular Biology 13, 549-554 (2006).
14. Hall. M. A. et al. High-resolution dynamic mapping of histone-DNA interactions in a nucleosome. Nature Structural & Molecular Biology 16, 124-129 (2009).
15. Killian, J. L., Ye, F. & Wang, M. D. Optical Tweezers: A Force to Be Reckoned With. Cell 175, 1445-1448 (2018).
16. Dagdas, Y. S., Chen, J. S., Sternberg, S. H., Doudna, J. A. & Yildiz, A. A conformational checkpoint between DNA binding and cleavage by CRISPR-Cas9. Science Advances 3 (2017).
17. Zhang, L. et al. Conformational Dynamics and Cleavage Sites of Cas12a Are Modulated by Complementarity between crRNA and DNA. iScience 19, 492-503 (2019).
18. Zhang, Q. et al. The post-PAM interaction of RNA-guided spCas9 with DNA dictates its target binding and dissociation. Science Advances 5, 1-11 (2019).
19. Szczelkun, M. D. et al. Direct observation of R-loop formation by single RNA-guided Cas9 and Cascade effector complexes. Proceedings of the National Academy of Sciences of the United States of America 111, 9798-9803 (2014).
20. Cofsky, J. C. et al. CRISPR-Cas 12a exploits R-loop asymmetry to form double-strand breaks. eLife 9, e55143 (2020).
21. Jinek, M. et al. Structures of Cas9 Endonucleases Reveal RNA-Mediated Conformational Activation. Science 343, 1247997 (2014).
22. Swarts, D. C., van der Oost, J. & Jinek, M. Structural Basis for Guide RNA Processing and Seed-Dependent DNA Targeting by CRISPR-Cas12a. Molecular cell 66, 221-233.e4 (2017).
23. Huai, C. et al. Structural insights into DNA cleavage activation of CRISPR-Cas9 system. Nature Communications 8, 1375 (2017).
24. Jin, J. et al. Synergistic action of RNA polymerases in overcoming the nucleosomal barrier. Nature Structural & Molecular Biology 17, 745-752 (2010).
25. Inman, J. T. et al. DNA Y Structure: A Versatile, Multidimensional Single Molecule Assay. Nano Letters 14, 6475-6480 (2014).
26. Le, T. T. et al. Mfd Dynamically Regulates Transcription via a Release and Catch-Up Mechanism. Cell 172, 344-357.e15 (2018).
27. Park, J.-S. & Roberts, J. W. Role of DNA bubble rewinding in enzymatic transcription termination. Proceedings of the National Academy of Sciences of the United States of America 103, 4870 (2006).
28. Komissarova, N., Becker, J., Solter, S., Kireeva, M. & Kashlev, M. Shortening of RNA: DNA hybrid in the elongation complex of RNA polymerase is a prerequisite for transcription termination. Molecular Cell 10, 1151-1162 (2002).
29. Park, J.-S., Marr, M. T. & Roberts, J. W. E. coli transcription repair coupling factor (Mfd protein) rescues arrested complexes by promoting forward translocation. Cell 109, 757-767 (2002).
30. Miao, C., Zhao, H., Qian, L. & Lou, C. Systematically investigating the key features of the DNase deactivated Cpf1 for tunable transcription regulation in prokaryotic cells. Synthetic and Systems Biotechnology 4. 1-9 (2019).
31. Clarke, R. et al. Enhanced Bacterial Immunity and Mammalian Genome Editing via RNA-Polymerase-Mediated Dislodging of Cas9 from Double-Strand DNA Breaks. Molecular Cell 71. 42-55.e8 (2018).
32. Epshtein, V., Toulme, F., Rahmouni, A. R., Borukhov, S. & Nudler, E. Transcription through the roadblocks: the role of RNA polymerase cooperation. The EMBO journal 22, 4719-4727 (2003).
33. Ma, J., Bai, L. & Wang, M. D. Transcription under torsion. Science 340, 1580-3 (2013).
34. Kotlajich, M. V. et al. Bridged filaments of histone-like nucleoid structuring protein pause RNA polymerase and aid termination in bacteria. Elife 4 (2015).
35. Nudler, E., Mustaev, A., Lukhtanov. E. & Goldfarb, A. The RNA-DNA hybrid maintains the register of transcription by preventing backtracking of RNA polymerase. Cell 89, 33-41 (1997).
36. Komissarova, N. & Kashlev, M. Transcriptional arrest: Escherichia coli RNA polymerase translocates backward, leaving the 3′ end of the RNA intact and extruded. Proceedings of the National Academy of Sciences 94, 1755 (1997).
37. Marr, M. T. & Roberts, J. W. Function of transcription cleavage factors GreA and GreB at a regulatory pause site. Mol Cell 6, 1275-85 (2000).
38. Stepanova, E., Wang, M., Severinov, K. & Borukhov, S. Early transcriptional arrest at Escherichia coli rpIN and ompX promoters. J Biol Chem 284, 35702-13 (2009).
39. Strobel, E. J. & Roberts, J. W. Regulation of promoter-proximal transcription elongation: enhanced DNA scrunching drives lambdaQ antiterminator-dependent escape from a sigma70-dependent pause. Nucleic Acids Res 42, 5097-108 (2014).
40. Howan, K. et al. Initiation of transcription-coupled repair characterized at single-molecule resolution. Nature 490, 431-434 (2012).
41. Portman, J. R., Brouwer, G. M., Bollins, J., Savery, N. J. & Strick, T. R. Cotranscriptional R-loop formation by Mfd involves topological partitioning of DNA. Proceedings of the National Academy of Sciences 118, e2019630118-e2019630118 (2021).
42. Le, T. T. & Wang, M. D. Molecular Highways-Navigating Collisions of DNA Motor Proteins. Journal of Molecular Biology 430, 4513-4524 (2018).
43. Haines, N. M., Kim, Y. I., Smith, A. J. & Savery, N. J. Stalled transcription complexes promote DNA repair at a distance. Proc Natl Acad Sci USA 111, 4037-42 (2014).
44. Graves, E. T. et al. A dynamic DNA-repair complex observed by correlative single-molecule nanomanipulation and fluorescence. Nat Struct Mol Biol 22, 452-7 (2015).
45. Ho, H. N., van Oijen, A. M. & Ghodke, H. The transcription-repair coupling factor Mfd associates with RNA polymerase in the absence of exogenous damage. Nature Communications 9, 1570 (2018).
46. Ma, H. H. et al. Multiplexed labeling of genomic loci with dCas9 and engineered sgRNAs using CRISPRainbow. Nature Biotechnology 34, 528-530 (2016).
47. Myers, S. A. et al. Discovery of proteins associated with a predefined genomic locus via dCas9-APEX-mediated proximity labeling. Nature Methods 15, 437-+ (2018).
48. Peters, J. E., Makarova, K. S., Shmakov, S. & Koonin, E. V. Recruitment of CRISPR-Cas systems by Tn7-like transposons. Proc Natl Acad Sci USA 114, E7358-E7366 (2017).
49. Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
50. Petassi, M. T., Hsieh, S. C. & Peters, J. E. Guide RNA Categorization Enables Target Site Choice in Tn7-CRISPR-Cas Transposons. Cell 183, 1757-1771 e18 (2020).
51. Richardson, C. D., Ray, G. J., DeWitt, M. A., Curie, G. L. & Com, J. E. Enhancing homology-directed genome editing by catalytically active and inactive CRISPR-Cas9 using asymmetric donor DNA. Nature Biotechnology 34, 339-344 (2016).
52. Yeh, C. D., Richardson, C. D. & Corn, J. E. Advances in genome editing through control of DNA repair pathways. Nature Cell Biology 21, 1468-1478 (2019).

### Methods

### Protein Purification

E. coli RNAP was purified using tagged purification 26,53,54 Briefly, RNAP was expressed at low levels in 5a-competent E. coli (Invitrogen, 18265-017) transformed with the plasmid pKA1 in Superbroth (25 g/L Tryptone (Sigma, T2559), 15 g/L yeast extract (Sigma, Y1626), 5 g/L NaCl (Sigma, S3014)) with 100 μg/mL ampicillin (Sigma, A0166) for 4 hours until A600 nm reached 2.1. Cells were induced with IPTG (RPI, 156000-50) to a final concentration of 1 mM for 4 hours. Cells were lysed and sonicated on ice in small aliquots (<20 mL). with a macro tip on a Branson Sonifier 250 with 60% duty cycle. Sonicated cells were centrifuged to pellet cell debris and the pellet was discarded. To precipitate nucleic acids and their bound proteins out of solution, cleared 5% (w/v) polyethyleneimine (PEI) pH 7.9 (made from 50% stock; Sigma, P3143) was slowly added to the supernatant to a final concentration of 0.4% (w/v). The DNA with bound RNAP was pelleted from the solution, washed five times with a buffer containing 350 mM NaCl (J. T. Baker, 4058-01), and RNAP was eluted from the PEI and DNA with a buffer containing 1 M NaCl. The eluted RNAP was purified to homogeneity with chromatography using three columns: a HiPrep Heparin FF 16/10 column (GE Healthcare, 28-9365-49), a HiPrep 26/60 Sephacryl S-300 HR column (GE Healthcare, 17-1196-01), and a QIAGEN Ni-NTA Superflow column (Qiagen, 30410). Fractions that contained holo-RNAP were pooled, concentrated, and dialyzed into RNAP storage buffer (50 mM Tris-HCl pH 8.0 (J. T. Baker, 4103-01 & 4109-01), 100 mM NaCl, 1 mM EDTA (Invitrogen, 15508-013) 50% (v/v) glycerol (J. T. Baker, 4043-00), and 1 mM DTT (Invitrogen, 15508-013)) and ultimately stored at −20° C.

E. coli greB was purified using tagged purification⁵⁴. Briefly, Plasmid pES3, encoding GreB-6×His in pET-28b (+) (4), was transformed into BL21 (DE3) (Invitrogen, 44-0049) cells for protein overexpression. Cells were grown at 37° C. in Luria Broth (LB) (Affymetrix, 75854) with 50 μg/ml of added Kanamycin (Sigma, K0254) at 37° C. until the OD600 nm was between 0.6-0.8, induction was then carried out with 1 mM IPTG (Roche, 10724815001). After 3 hrs at 37° C., cells were harvested by centrifugation and stored at −80° C. To purify GreB, cells were thawed on ice and re-suspended in GreB Lysis Buffer (50 mM Tris-HCl (Fisher, BP154 & BP153) pH 6.9, 500 mM NaCl (Fisher, BP358), and 5% v/v glycerol (Fisher, BP229),) using lysozyme (300 μg/ml) (Sigma, 10837059001) EDTA-free protease-inhibitor cocktail (Roche, 11873580001). The cells were placed on ice for 1 hour and then briefly sonicated for more complete lysis. The extract was centrifuged (24,000 g, 20 min at 4° C.) and twice passed through a 0.45-μm filter. An Ni-NTA agarose (Invitrogen, R90115) column was used for GreB isolation and GreB Lysis Buffer with 200 mM imidazole was used for elution. The eluate was then run on a Superdex 200 column (Cytivia, 28990944) with Elution Buffer (10 mM Tris-HCl pH 8.0, 500 mM NaCl, 1 mM DTT, 1 mM EDTA, and 5% v/v glycerol). Dialysis was performed into GreB storage buffer (10 mM Tris-HCl pH 8.0, 200 mM NaCl, 1 mM DTT, 1 mM EDTA, and 50% v/v glycerol), and stored at −80° C. after a flash freeze n liquid nitrogen.

E. coli Mfd was purified using tagged purification⁵⁵. Briefly, a pET plasmid was used to overexpress Eco Mfd with its N-terminus His6-tagged. This plasmid was transformed via heat shock at 42° C. for 40 seconds into Rosetta (DE3) pLysS cells (Novagen, 70956-M). 1 mM isopropyl β-D-1-thiogalactopyranoside (IPTG) (Goldbio, I2481C) was added to cells (O. D. 0.67) for 4 hours at 30° C. to induce protein expression. For harvesting, cells were centrifuged and pellets were resuspended in a lysis buffer (50 mM Tris (MP, 103133), pH 8.0, 500 mM NaCl (Fisher, S271-500), 15 mM imidazole (MP, 02102033—CF), 10% (v/v) glycerol (Fisher, BP2294), 2 mM β-mercaptoethanol (β-ME) (Sigma, M6250), 1 mM PMSF (Sigma, P7626), and protease inhibitor cocktail (complete, EDTA-free; Roche, COEDTAF-RO) and subsequently lysed on a french press. Lysate was flown over a Ni²⁺-charged Hitrap IMAC column (Cytiva, 17524802) and eluted over the course of a 0-200 mM imidazole gradient. Post-nickel column dialysis was performed in a buffer containing 20 mM Tris, pH 8.0, 100 mM NaCl, 10% (v/v) glycerol, 5 mM EDTA (Sigma, E5134), and 10 mM β-ME, and the dialyzed sample was loaded onto a Hitrap Heparin column (Cytiva, 28-9893-35). Elution was performed over the course of a 100 mM 2 M NaCl gradient, and the resulting sample was further purified on a HiLoad 16/600 Superdex200 size exclusion chromatography column (Cytiva, 29-9893-35) in a buffer containing 20 mM Tris, pH 8.0, 500 mM NaCl, and 10 mM DTT (Goldbio, DTT10). Glycerol was added to the purified Mfd to reach a final concentration of 20% (v/v), sample was flash frozen in liquid N₂, and finally stored at −80° C.

gRNA Preparation

Cas9 sgRNAs were custom synthesized by Sigma Aldrich, and purified by 8% denaturing Urea polyacrylamide gel electrophoresis (Urea-PAGE) similar to previous descriptions^{16,56} Cas12a gRNA was prepared by cloning the Cas12a gRNA sequence⁵⁷ (Supplementary Table. 2) into a pUC19 plasmid, containing a T7 promoter and a downstream HDV ribozyme sequence⁵⁸, by site directed mutagenesis. T7 transcription (IVT) templates were generated from the cloned plasmids via PCR with Q5 DNA polymerase (NEB, M0491). In vitro transcription was performed for each template by incubation with T7 RNAP (NEB, M0251) at 37° C. for 3 hours, followed by incubation at 65° C. for 20 min to promote ribozyme cleavage and leave a 3′ cyclic phosphate. Products were dephosphorylated with T4 PNK (NEB, M0201) and purified by urea-PAGE.

### Single-Molecule DNA Unzipping Templates

Single-molecule DNA unzipping templates were generated from a pRL574 plasmid, which contains a T7A1 promoter. The PAM-Distal Cas9 template was identified in pRL574, 309 bp from the +20. To generate templates for the remaining three templates (Cas9 PAM-proximal, Cas12a PAM-Proximal, and Cas12a PAM-Distal), a ˜60 bp region of pRL574 was modified via site directed mutagenesis using a protocol from NEB and Q5 DNA polymerase. For each template, we substituted a 63 or 64 bp DNA segment at 290 bp from the +20 for Cas9 or both Cas12a templates, respectively. The substituted DNA segment contained the relevant target sequence and PAM as well as 20 bp of conserved flanking DNA on either side.

Four DNA unzipping segments were amplified by PCR, digested with DraIII (NEB, R3510) leaving a ssDNA overhang (TAG), and purified by 0.8% agarose gel electrophoresis These templates were used as transcription templates, and for PAM-distal dCas and upstream RNAP mapping. Two additional reversed unzipping segments were used for PAM-proximal dCas and downstream PTC mapping and generated by PCR and digested with AlwNI (NEB, R0514). These DNA segments were then each ligated to a pair of dsDNA arms containing a CTA overhang at their junction^{25,26}. Both DNA arms were amplified by PCR from pBR322 (NEB, N3033) and digested by BamHI. One arm was end-labeled with biotin—and the other with digoxigenin through separate Klenow reactions with biotin-14-dATP (Invitrogen, 19524016) and digoxigenin-11-dUTP (Roche, 11093088910), respectively. Each arm was digested with BsmBi-V2 (NEB, R0739S), ligated to an annealed adapter oligo, and gel purified. Finally, the arms were annealed to each other at an equimolar ratio to create y-arm adapters suitable for ligation of an unzipping segment.

### Protein Complex Formation for Single-Molecule Experiments

Paused transcription complex (PTC) was formed in bulk on an unzipping template which contained a promoter in the unzipping segment. The complex was paused at the A20 position via nucleotide depletion^{24,26} Briefly, 10 nM DNA template was mixed with 50 nM RNAP in the presence of 250 μM ApU (Dharmacon, custom synthesis), 50 μM GTP (Roche, 11140957001), ATP (Roche, 11140965001) and CTP (Roche, 11140922001), 1 U/μl of Superase-in (Invitrogen, AM2694) in transcription buffer (TB, 25 mM Tris-Cl (Fisher, BP154 & BP153) pH 8, 100 mM KCl (P333), 4 mM MgCl₂ (Invitrogen, AM9530G), 1 mM DTT (Invitrogen, 15508-013), 3% glycerol (Fisher, BP229), 150 μg/ml AcBSA (Invitrogen, AM2614). For high resolution TEC mapping experiments, the mixture also contained 1 mM 3′-deoxy-UTP (Trilink, N-3005)⁵⁹ which paused the complex at U21. For all experiments, the mixture was incubated at 37° C. for 30 min and then briefly placed on ice. The mixture was quickly diluted 1:100 and immediately introduced into a prepared sample chamber. To form dCas-gRNA complex. 50 nM Cas9-sgRNA or 100 nM Cas12a-gRNA was denatured in RNA storage solution (Invitrogen, AM7001) at 80° C. for 1.5 min and then placed on ice. 75 nM Sp-dCas9 (NEB, M0652) or 300 nM As-dCas12a (IDT, off catalog) along with 1×TB was then added. The mixture was incubated at 37° C. for 10 min and then placed on ice until introduction into a prepared sample chamber. dCas-gRNA complex was later introduced into a single molecule sample chamber to allow dCas binding to DNA as described below.

### Single-Molecule Experimental Procedures

For all unzipping assays, DNA tethers were formed in a sample chamber consisting of a cleaned glass coverslip as previously described²⁴⁻²⁶. Anti-digoxigenin (Vector Labs, MB-7000) in TB at 16.7 μg/ml was introduced into the chamber, allowed to incubate for 10 minutes at RT, and replaced with 65 μl TB with 10 mg/ml casein (Sigma, C8654). After 10 min at RT, 5 pM DNA template was introduced, allowed to incubate for 5 min at RT, and later replaced with 90 μl TB. Finally, 0.5 pM of 489 nm streptavidin coated polystyrene beads in TB with 1 mg/ml casein was introduced and incubated for 10 min and RT. The buffer was replaced with 80 μl TB. This resulted in DNA templates tethered between the surface of a coverslip via a dioxygenin (dig) and anti-dig connection and a 489 nm bead via a biotin and streptavidin connection (FIG. 1a).

dCas-DNA complexes were formed on DNA tethers by introducing 75 μl of prepared dCas-gRNA complexes (25 pM dCas9-sgRNA or 200 pM dCas12a-sgRNA) and incubating for 10 min before replacing the chamber buffer with 90 μl of TB. For inverted repeat gRNAs, 37.5 pM dCas9-sgRNA was introduced. For 3-bp mismatched guides, 50 pM was introduced to ensure a high fraction of bound dCas9 (>90%).

For roadblock assays, PTCs and dCas-DNA complexes were formed as described using the appropriate unzipping template for the selected dCas target (Supplementary Table 2). Free dCas proteins were removed by flushing the sample chamber with 90 μl of TB. Subsequently, occupancy of each bound protein was assessed via unzipping ˜40 tethers. For transcription resumption, 75 μl of TB buffer supplemented with 1 mM NTP each (UTP; Roche, 11140949001), and 1 mM MgCl₂ was introduced into the sample chamber. The transcription reaction was chased for 135 s before being quenched by introducing 120 ul of TB with 4 mM Mg²⁺ into the chamber. For Mfd translocase experiments, 75 ul of 166 nM Mfd with 2 mM ATP and 4 mM Mg²⁺ in TB was introduced into the sample chamber. After 480 s, the reaction was quenched by introducing 75 μl of TB with 1 mM ATPyS (Sigma-Aldrich, A1388) and 5 mM Mg²⁺. After quenching, the bound proteins were assayed by unzipping 60 or 80 tethers for transcription or translocation reactions, respectively.

### Optical Trapping Measurements

We used a surface based optical trap setup⁶⁰ (FIG. 1a). For FIG. 1 and FIG. 4b, tethers were unzipped at a loading rate of 8 pN/s. For the remaining experiments, tethers were unzipped at a constant velocity of 500 nm/s. Data for all assays was acquired at 10 kHz and decimated with averaging to 1 kHlz. Raw force and extension data were used to obtain the number of base-pairs unzipped via dsDNA and ssDNA elastic parameters^{25,61}. The force versus number of base-pairs unzipped was then aligned to the expected unzipping theory curve to increase the accuracy and precision to locate bound protein interactions²⁶. Data acquisition and conversion were performed using Custom Lab View 7 software, and all downstream analyses were performed using Custom Matlab 7 Code.

The force peak position of a protein bound to DNA was identified as the location of a vertical force rise that deviated from the theoretical force versus number of base-pairs unzipped. Subsequent to transcription reactions, some force peaks near the RNAP showed a small but distinct tether shortening event. This was attributed to the nascent transcript partially annealing to the exposed single stranded DNA. For traces that had this detectable shift, this slight shortening was corrected for in the location of the dCas9 peak.

All optical trapping measurements were performed in a temperature-controlled room at 23.3° C. However, the temperature increased slightly to an estimated 25° C. owing to local laser trap heating⁶². All reactions were also carried out at the room temperature of 23.3° C.

### Calculation of Transcription Read-Through of the Single-Molecule Experiments

To accurately quantify the rate of dCas read-through by a translocase, we need to take into account of the following considerations: (1) the initial state of the traces might vary slightly from sample chamber to sample chamber, (2) proteins initially bound to DNA might dissociate through a non-collision mechanism, and (3) some translocases were inactive or could not reach the collision site. Therefore, to calculate read-through rate after these considerations, we have used conditional probabilities to determine how each category of traces changed after chasing.

Before chasing, we have classified traces into one of four fractions. For a given sample chamber, the fraction of PTC at A20 and a dCas (F_{A20,dCas i}) was always the dominating fraction, representing typically 90% of traces, and was measured for each sample chamber. The remaining traces were categorized at PTC only, dCas only, or neither, with fractions denoted as F_{A20 only i}, F_{acas only i}, and F_{Nak i}, respectively. These three minor fractions also contributed to various final observed fractions and their contributions must be accounted for (FIG. 5).

Additionally, we found that a small, but significant, fraction of the TEC and bound dCas9 dissociated during the course of the experiment in the absence of any translocase activity. We accounted for this by including the probability of TEC and dCas dissociating through a non-collision mechanism as P_{RNAP_diss} and P_{acas diss}, respectively (FIG. 2).

After chasing, traces were categorized into one of seven fractions. Traces that showed a TEC that had not yet reached the bound dCas were classified as either with a dCas (F_{TEC_up,dCas_f}) and without dCas (F_{TEC_up_only_f}). We classified a trace with a TEC that has not reached dCas as those with a detected TEC force peak >60 bp upstream from the dCas target site. Traces with a TEC <60 bp upstream from dCas site and with a dCas present were categorized as having had a dCas-RNAP collision (F_{Coll f}). Traces with a TEC <60 bp upstream from the dCas site but without a dCas detected were categorized as RNAP having removed dCas but then being unable to read-through (F_{acas rem_f}). Lastly, we also categorized traces with a TEC downstream of the dCas binding site without dCas being present (F_{TEC_dn_f}), traces with no bound proteins (F_{Nak f}), or traces which consisted of dCas only with no TEC detected (F_{acas_f})

Due to the heterogeneities in the TEC population and bound dCas, not all TEC complexes would encounter a bound dCas. We refer to the probability that an RNAP initially escaped the A20 translocated toward, and reached a bound dCas, as the probability of being collision competent, P_{Coll_comp}. We can determine this probability from probability of TEC being collision incompetent, that is, the probability that a TEC was present at A20 or upstream of the dCas, given RNAP did not dissociate through a non-collision mechanism. P_{Coll_comp} can be calculated as:

$${P}_{Coll\_comp} = 1 - \frac{{F}_{TEC\_up , dCas\_f} + {F}_{TEC\_up \_only \_f}}{( {F}_{A20 , dCas\_i} + {F}_{A20\_only \_i} ) ( 1 - {P}_{RNAP\_diss} )}$$

To determine the probability that a TEC was able to read-through a dCas, given that the TEC was collision competent and neither protein dissociated due to a non-collision mechanism, we start with the post-chase naked DNA (F_{Nak f}) and RNAP downstream (F_{TEC_dn f}) traces, and then take into account other pathways that also contributed to those two final observations. This gives the following equation for P_{Read-through}

$${P}_{Read - through} = [ {F}_{Nak\_f} + {F}_{TEC\_dn \_f} - {F}_{Nak\_i} - {F}_{A 20 , dCas\_i} ( {P}_{Coll\_comp} ( 1 - {P}_{RNAP\_diss} ) + {P}_{RNAP\_diss} ) {P}_{dCas\_diss} - {F}_{A 20 \_only \_i} ( {P}_{Com\_comp} ( 1 - {P}_{RNAP\_diss} ) + {P}_{RNAP\_diss} ) - {F}_{dCas\_only \_i} {P}_{dCas\_diss} ] / [ {F}_{A 20 , dCas\_i} {P}_{Coll\_comp} ( 1 - {P}_{RNAP\_diss} ) ( 1 - {P}_{dCas 9\_ diss} ) ]$$

Similarly we can find the probability that a TEC will remove the dCas from the DNA by also including the fraction of traces where TEC was found to have removed dCas but was not able to read-though (F_{acas rem_f}). This results in the following equation for P_{Removal}:

$${P}_{Removal} = [ {F}_{Nak\_f} + {F}_{TEC\_dn \_f} + {F}_{dCas rem\_f} - {F}_{Nak\_i} - {F}_{A 20 , dCas\_i} ( {P}_{Coll\_comp} ( 1 - {P}_{RNAP\_diss} ) + {P}_{RNAP\_diss} ) {P}_{dCas\_diss} - {F}_{A 20 \_only \_i} ( {P}_{Com\_comp} ( 1 - {P}_{RNAP\_diss} ) + {P}_{RNAP\_diss} ) - {F}_{dCas\_only \_i} {P}_{dCas\_diss} ] / [ {F}_{A 20 , dCas\_i} {P}_{Coll\_comp} ( 1 - {P}_{RNAP\_diss} ) ( 1 - {P}_{dCas 9\_ diss} ) ]$$

For the Mfd collision assays of FIG. 3, the calculation of move-through is nearly identical, however, we must replace P_{RNAP_diss} with the observed non-collision related dissociation of Mfd before translocation (P_{Mfd_diss}) (FIG. 7c). We also define the cutoff for Mfd being collision competent to be <70 bp as this corresponds with the approximate footprint of the complex²⁶. Finally, we did not observe a population of stalled Mfd near the dCas binding after dCas removal, and the removal efficiency is identical to the move-through efficiency.

Bulk transcription assays Bulk transcription assays were done using P-32 labelled RNA, separated by Urea-PAGE gel electrophoresis^{26,53,63}. Four 5′-biotinylated DNA templates, each containing the T7A1 promoter and a dCas binding site, were amplified from the pRL574 variants using Taq Polymerase (NEB, M0273). The templates were bound to streptavidin coated magnetic beads (NEB, S1420) at a concentration of 100 nM and mixed by rotation for 12 hours at 4° C. Paused transcription complexes (PTCs) were made in a similar fashion as noted above for single-molecule assays by combining 20 nM bead-bound DNA, 100 nM RNAP, 50 μM CTP, 50 μM ATP, 30 uCi of a-32P GTP (Perkin-Elmer, BLU006H250UC), 250 μM ApU, and 1 U/μl Superase-in and incubating for 30 min at 37° C. PTCs were then immediately washed three times with TB. A magnetic tube rack was used to pull down PTCs, and the pellet was washed and resuspended in TB. dCas-gRNA complexes were formed similarly to single-molecule assays, added to the washed PTCs (40 nM for dCas9, 250 nM for dCas12a), and incubated at 37° C. for 10 min. Resulting PTCs and dCas complexes were then washed with TB as before to remove free dCas-gRNA. Finally, TECs primed for collision with bound dCas-gRNA were chased by adding 1 mM NTPs with or without 1 μM GreB in TB with 5 mM MgCl₂ for 135 s. The reaction was quenched and transcripts were released from TECs by adding 1×RNA loading dye (NEB, B0363) and 25 mM EDTA (MP, 194822). Magnetic beads were pulled down using a magnetic rack. The supernatant containing the transcript was removed, heated to 95° C. for 10 min, and then immediately loaded onto a 20 cm 6% urea-PAGE gel pre-run to 55° C. using a Protean Xi Cell (Bio-Rad). The gel was dried using a Model 583 gel dryer (Bio-Rad), exposed to a phosphor screen (FujiFilm) for 12 hours, and scanned on a Typhoon 700 Imager (Cytiva). Images were linearized using ImageJ, and lane profiles were analyzed using Matlab.

### Method Examples—Only References

53. Adelman, K. et al. Single molecule analysis of RNA polymerase elongation reveals uniform kinetic behavior. Proceedings of the National Academy of Sciences 99, 13538-13543 (2002).
54. Ma, J. et al. Transcription factor regulation of RNA polymerase's torque generation capacity. Proceedings of the National Academy of Sciences 116, 2583 (2019).
55. Kang. J. Y. et al. Structural basis of transcription arrest by coliphage HK022 nun in an Escherichia coli rna polymerase elongation complex. eLife 6, 1-20 (2017).
56. Jiang, F., Zhou, K., Ma, L., Gressel, S. & Doudna, J. A. A Cas9-guide RNA complex preorganized for target DNA recognition. Science 348, 1477-1481 (2015).
57. Yamano, T. et al. Crystal Structure of Cpf1 in Complex with Guide RNA and Target DNA. Cell 165, 949-962 (2016).
58. Battaglia, R. A., Price, I. R. & Ke, A. Structural basis for guanidine sensing by the ykkC family of riboswitches. RNA (New York. N.Y.) 23, 578-585 (2017).
59. Kang. J. Y. et al. Structural basis for transcription complex disruption by the Mfd translocase. eLife 10 (2021).
60. Brower-Toland, B. D. et al. Mechanical disruption of individual nucleosomes reveals a reversible multistage release of DNA. Proceedings of the National Academy of Sciences 99, 1960-1965 (2002).
61. Li, M. & Wang, M. D. Unzipping Single DNA Molecules to Study Nucleosome Structure and Dynamics. Nucleosomes, Histones & Chromatin, Pt B 513, 29-58 (2012).
62. Peterman, E. J., Gittes, F. & Schmidt, C. F. Laser-induced heating in optical traps. Biophys J 84, 1308-16 (2003).
63. Adelman, K. et al. Molecular mechanism of transcription inhibition by peptide antibiotic microcin J25. Molecular Cell 14, 753-762 (2004).
64. Anders, C., Niewoehner, O., Duerst, A. & Jinek, M. Structural basis of PAM-dependent target DNA recognition by the Cas9 endonuclease. Nature 513, 569-573 (2014).
65. Swarts, D. C. & Jinek, M. Mechanistic Insights into the cis—and trans-Acting DNase Activities of Cas 12a. Molecular Cell 73, 589-600.e4 (2019).
66. Wang, D. et al. Discontinuous movements of DNA and RNA in RNA polymerase accompany formation of a paused transcription complex. Cell 81, 341-350 (1995).

1. A modified guide RNA for use with a CRISPR Cas protein, the modified guide RNA comprising at its 5′ or 3′ end at least 5 nucleotides that comprise an inverted repeat sequence having a segment targeted to a spacer sequence in DNA, and wherein the inverted repeat sequence is configured so that it can concurrently be hybridized to the spacer sequence and to the complementary strand of the DNA comprising the spacer sequence when in the presence of the DNA and the Cas protein.

2. The modified guide RNA of claim 1, wherein the inverted repeat comprises 5, 6, or 7 nucleotides.

3. The modified guide RNA of claim 1, wherein the CRISPR Cas protein comprises a Cas9 protein or a dCas9 protein.

4. The modified guide RNA of claim 1, wherein the at least 5 nucleotides are at the 5′ end of the modified guide RNA.

5. A ribonucleoprotein comprising a CRISPR Cas protein and a modified guide RNA according to claim 1.

6. A method comprising introducing into cells a modified guide RNA comprising at its 5′ or 3′ end at least 5 nucleotides that comprise an inverted repeat sequence having a segment targeted to a spacer sequence in DNA, and wherein the inverted repeat sequence is configured so that it can concurrently be hybridized to the spacer sequence and to the complementary strand of the DNA comprising the spacer sequence, and a CRISPR Cas protein that is functional with the guide RNA, such that a complex comprising the guide RNA, the DNA and the Cas protein forms within the cell.

7. The method of claim 6, wherein the inverted repeat comprises 5, 6, or 7 nucleotides.

8. The method of claim 7, wherein the complex is associated with the DNA for a period of time that is longer than a period that time relative to a complex comprising the Cas protein and an unmodified guide RNA targeting the same spacer.

9. The method of claim 8, wherein the DNA comprises a protospacer adjacent motif (PAM) that is at the 5′ end of the spacer sequence.

10. The method of claim 6, wherein the CRISPR Cas protein comprises a Cas9 protein or a dCas9 protein.

11. The method of claim 10, wherein the guide RNA and the CRISPR Cas protein are administered to the cells as a ribonucleoprotein complex.

12. The method of claim 11, wherein the cells are eukaryotic cells.

13. The method of claim 12, wherein the Cas protein comprises a nuclear localization signal.

14. A expression vector encoding a guide RNA of claim 1.

15. A cDNA corresponding to a guide RNA of claim 1.

16. A complex comprising a double stranded DNA molecule and a modified guide RNA comprising at its 5′ end or 3′ end at least 5 nucleotides that comprise an inverted repeat sequence having a segment targeted to a spacer sequence in the double stranded DNA molecule, and wherein the inverted repeat sequence is concurrently hybridized to the spacer sequence and to the complementary strand of the double stranded DNA, and a CRISPR Cas protein that is functional with the modified guide RNA.

17. The double stranded DNA molecule of claim 16, wherein the CRISPR Cas protein is a Cas9 protein or a dCas9 protein.

18. The double stranded DNA molecule of claim 16, wherein the segment at the 5′ end of the guide RNA comprises 5, 6, or 7 nucleotides.

19. The double stranded DNA complex of claim 18, wherein the complex is present within a cell.

20. The double stranded DNA complex of claim 19, wherein the cell comprises a eukaryotic cell.
